(19) United States
(12) Patent Application Publication
(10) Pub. No.: US 2016/0095850 A1
(43) Pub. Date: Apr. 7, 2016

(54) METHODS AND COMPOSITIONS FOR TREATMENT OF HIV INFECTION

(71) Applicant: COOPER HUMAN SYSTEMS LLC, Nashua, NH (US)

(72) Inventors: Kenneth G. Cooper, Nashua, NH (US); Mark S. De Souza, Bangkok (TH); Keith Eubanks, Arlington, MA (US); David H. Starr, Amherst, NH (US); John D. Kapson, Hollis, NJ (US); Hua Yang, Concord, MA (US)

(21) Appl. No.: 14/893,359

(22) PCT Filed: Apr. 24, 2014
(86) PCT No.: PCT/US2014/035354

§ 371 Date: Nov. 23, 2015

Related U.S. Application Data
(60) Provisional application No. 61/827,314, filed on May 24, 2013.
(60) Provisional application No. 61/866,865, filed on Aug. 16, 2013.

(51) Int. Cl. A61K 31/46; A61K 45/06; A61K 38/20; A61K 31/4706; A61K 31/167

(52) U.S. Cl. CPC A61K 31/46; A61K 31/4706; A61K 31/167; A61K 38/2086; A61K 45/06

(57) ABSTRACT

Methods and compositions for treatment of human immunodeficiency virus (HIV) infections have been developed which dampen immune activation with a bias more on the CD4 T cells relative to the CD8 T cell response, inhibit HIV replication, reactivate latent HIV, and inhibit infection of cells by HIV. Pushing latent HIV into active infections with hindrance of cell infection by the reactivated HIV can substantially reduce the number of cells infected with HIV and the viral load of HIV, which is not achieved using just the combination of ART and compounds which activate latent HIV. The methods involve administering to an HIV-infected subject three or more compounds which collectively dampen immune activation with a bias more on the CD4 T cells relative to the CD8 T cell response, inhibit HIV replication, reactivate latent HIV, and inhibiting infection of CD4 T cells by HIV.

### CROSS REFERENCE TO RELATED APPLICATIONS

This application claims priority to U.S. Ser. No. 61/827,314 filed May 24, 2013 by Kenneth G. Cooper, Mark S. De Souza, Keith Eubanks, John D. Kapson, and Hua Yang and to U.S. Ser. No. 61/866,865 filed Aug. 16, 2013, by Kenneth G. Cooper, Mark S. De Souza, Keith Eubanks, David H. Starr, John D. Kapson, and Hua Yang.

### FIELD OF THE INVENTION

The invention is in the general field of treatment of HIV infections and particularly in the field of treatment of latent HIV infections to maintain reduced viral load following cessation of drug treatment.

### BACKGROUND OF THE INVENTION

Human immunodeficiency virus (HIV) affects specific cells of the immune system, called CD4 cells, or T cells. Over time, HIV can destroy so many of these cells that the body cannot fight off infections and disease. HIV disease has a well-documented progression. Untreated, HIV is almost universally fatal because it eventually overwhelms the immune system—resulting in acquired immunodeficiency syndrome (AIDS). HIV treatment helps people at all stages of the disease, and treatment can slow or prevent progression from one stage to the next.

HIV progresses through three stages:

Acute infection: Within 2 to 4 weeks after infection with HIV, acute retroviral syndrome (ARS) or primary HIV infection, results in large amounts of HIV being produced in your body. The virus uses CD4 cells to make copies of itself and destroys these cells in the process. The amount of virus in the blood is very high during this stage. Eventually, the immune response will reduce the amount of virus to a stable level, and the CD4 count will begin to increase, but typically does not return to pre-infection levels.

Clinical latency (inactivity or dormancy): This period is sometimes called asymptomatic HIV infection or chronic HIV infection. During this phase, HIV is still active, but reproduces at very low levels, and the individual may not have any symptoms or get sick during this time. People who are on antiretroviral therapy (ART) may live with clinical latency for several decades. For people who are not on ART, this period can last up to a decade, but some may progress through this phase faster. Toward the middle and end of this period, the viral load begins to rise and the CD4 cell count continues to drop. This correlates with development of symptoms of HIV infection as the immune system becomes too weak to protect against other diseases and cancer.

AIDS (acquired immunodeficiency syndrome): This is the stage of infection that occurs when one becomes vulnerable to a range of bacterial, viral and fungal pathogens termed opportunistic infections. AIDS is defined as when the number of CD4 cells falls below 200 cells/mm³ blood. AIDS may also be diagnosed upon development of one or more opportunistic infections, regardless of the CD4 count. Without treatment, people who are diagnosed with AIDS typically survive about three years.

The HIV reservoir is established during primary infection. Administration of anti-retroviral therapy (“ART”) very early in acute infection seems to result in a low post-treatment HIV viral load, suggesting that aggressive treatment can decrease the size of the viral reservoir (Hocqueloux et al., 2010; Chun et al., J Infect Dis 2007; 195: 1762-64; Ananworanich et al., PLoS One 2012; 7: e33948; Archin et al., Proc. Natl. Acad. Sci. USA 2012; 109: 9523-28). Although early treatment can substantially reduce the size of the total reservoir, a stable population of latently infected CD4 T cells develops into the long-lived latent reservoir, and is unaffected by early combination ART (cART) (von Wyl et al., PLoS One 2011; 6: e27463). Most proviral HIV is detected in CD4+T lymphocytes in lymphoid tissue (Hufert et al., AIDS 1997; 11: 849-57; Stellbrink et al., AIDS 1997; 11: 1103-10). In blood, most proviral HIV is found in central memory and transitional memory T cells, which maintain the reservoir because of their intrinsic capacity to persist through homoeostatic proliferation and renewal (Chomont et al., Nat. Med. 2009; 15: 893-900). Other cellular reservoirs that might exist include naive CD4 T cells, monocytes and macrophages, astrocytes, microglial cells (Deeks et al., Nat. Rev. Immunol. 2012; 12: 607-14) and T stem cell memory cells (Buzon et al. Nat Med. 2014 February; 20(2):139-42). During long-term effective ART, a steady-state, low-level plasma HIV viral load can be achieved, typically from less than one to three copies of HIV per ml. (Palmer et al., Proc. Natl. Acad. Sci. USA 2008; 105: 3879-84). Chronic production of HIV from a stable reservoir of long-lived infected cells (the so-called latent reservoir) is probably the main source of this persistent HIV.

A prerequisite for the establishment of HIV latency is the integration of viral DNA into the host chromatin and epigenetic silencing of active viral transcription. The molecular mechanisms contributing to the silencing of latent HIV are complex (Karn and Stoltzfus, Cold Spring Harb. Perspect. Med. 2012; 2: a006916). Infected cells with replication-competent provirus are transcriptionally silenced by co-repressor complexes that include histone deacetylases, histone methyltransferases, and heterochromatin proteins. Active methylation of the long terminal repeat might also play a part (Van Duyne et al., J. Mol. Biol. 2011; 411: 581-96; Friedman et al., J. Virol. 2011; 85: 9078-89). Epigenetic silencing of a provirus can be reversed by agents that mobilize chromatin remodeling complexes to replace repressive complexes poised at the viral long terminal repeat (Hakre et al., FEMS Microbiol. Rev. 2012; 36: 706-16). Signals delivered through the T cell receptor (TCR-CD3) complex and CD28 co-stimulation can drive productive transcription, suggesting that physiological activation of memory CD4 T cells can lead to virus production in vivo (Rong and Perelson, PLoS Comput. Biol. 2009; 5: e1000533). Activated CD4 T cells are the most permissive target for HIV infection. How recently infected activated cells become long-lived latently infected resting memory cells is not fully understood. Many regulatory pathways designed to blunt the effect of cell activation are turned on during T cell activation, including the upregulation of negative regulators of T cell activation—for example, PD-1, CTLA-4, TRIM-3, LAG3, CD160, and 2B4 cell surface receptors. Cells expressing these receptors could be preferential reservoirs of HIV. In a cross-sectional study of long-term treated individuals, PD-1-expressing cells were enriched with latent HIV (Chomont et al., Nat Med 2009; 15: 893).

ART is one of the major medical successes in the era of AIDS. ART can provide indefinite viral suppression, restored immune function, improved quality of life, the near normalization of expected lifespan, and reduced viral transmission. However, ART does not eliminate viral reservoirs, and needs to be used indefinitely to keep AIDS at bay. ART is also expensive with potential short-term and long-term toxic effects. Despite virus control, HIV-associated complications persist, including a higher than normal risk of cardiovascular disease, cancer, osteoporosis, and other end-organ diseases. This increased risk might be due to the toxic effects of treatment or the consequences of persistent inflammation and immune dysfunction associated with HIV. Treatment approaches that eliminate persistent virus and do not need lifelong adherence to expensive and potentially toxic antiretroviral drugs are needed.

There are two general categories of a “cure” for HIV infection: a functional cure and a sterilizing cure. A functional cure is defined as an intervention that renders patients with progressive disease able to permanently control viral replication, thereby preventing clinical immunodeficiency and transmission (adapted from: Eisele E, Siliciano RF. Redefining the viral reservoirs that prevent HIV-1 eradication. Immunity. 2012 Sep. 21; 37(3):377-88). A functional cure suppresses viral replication for a pre-defined period of time in the absence of drug therapy, restores and stabilizes effective immune function, and decreases both HIV-induced inflammation (which could increase the risk of AIDS or non-AIDS morbidity) and, in those individuals that maintain stable low-level plasma viral loads, reduces the risk of virus transmission to others.

The World Health Organization (WHO) recommends first-line anti-retroviral therapy (“ART”) consist of two nucleoside reverse transcriptase inhibitors (NRTIs) plus a non-nucleoside reverse-transcriptase inhibitor (NNRTI). TDF+3TC (or FTC)+EFV as a fixed-dose combination is recommended as the preferred option to initiate ART (strong recommendation, moderate-quality evidence). If TDF+3TC (or FTC)+EFV is contraindicated or not available, one of the following options is recommended: AZT+3TC+EFV; AZT+3TC+NVP; or TDF+3TC (or FTC)+NVP

(strong recommendation, moderate-quality evidence).

As reported by Messiaen et al., PLoS One. 2013; 8(1):e52562 (Epub Jan. 9, 2013), an optimal regimen choice of antiretroviral therapy is essential to achieve long-term clinical success. Integrase inhibitors have been adopted as part of current antiretroviral regimens. However, integrase inhibitors combined with protease inhibitors do not result in a significant better virological outcome.

As most recently reviewed by Lewin, The Lancet, 381(9883):2057-2058 (15 Jun. 2013), there is still no cure for HIV, although a few cases of functional cures have been reported, one due to a naturally occurring mutation in the CCR5 gene, one in a newborn given immediate ART at birth, and a few people who were treated immediately upon infection. These are the exceptions. Current therapy is now focused on activating HIV from resting T cells. Activating latent virus might lead to death of the cell or make the virus ready for immune-mediated clearance. A range of drugs that modify gene expression, including viral gene expression, are in clinical trials in HIV-infected patients on ART. Two studies have reported that HIV latency can be activated with the histone deacetylase inhibitor Vorinostat. The frequency of HIV-cure related trials is increasing annually based on the findings of the VISCONTI cohort (Sáez-Cirión et al. PLoS Pathog. 2013 March; 9(3):e1003211. doi: 10.1371/journal.ppat.1003211. Epub 2013 Mar. 14. and the “Mississippi baby” treatment outcome (Persaud D, et al. N Engl. J. Med. 2014 Feb. 13; 370(7):678). Clinical trials include investigations of increasingly potent histone deacetylase inhibitors, and of gene therapy to eliminate the CCR5 receptor from patient-derived cells. HIV-cure-related trials raise many complex issues, given potentially toxic interventions to patients doing very well on ART, and needs careful assessment.

Rasmussen et al., Human Vaccines & Immunotherapeutics 9:4, 790-799 (April 2013), review all of the strategies proposed to eradicate HIV infection. Prolonged combination antiretroviral treatment (cART) has not led to eradication of HIV infection. Current research is focused on characterizing latent HIV reservoirs and understanding the intricate mechanisms that establish HIV latency and enable the virus to persist for decades evading host immune responses and potent cART. It is useful to distinguish between proviral latency, referring to the presence of replication competent but transcriptionally silent provirus within resting cells, and residual viremia, referring to the continuous existence of trace levels of extracellular HIV-RNA in plasma during suppressive cART. Whereas the pool of latently infected memory CD4+ T-cells is now the most well-defined latent HIV reservoir and presumably the primary obstacle to the eradication of HIV infection, the origin and significance of the residual viremia, in particular whether this is caused by on-going replication, is still debated.

Several therapeutic strategies are being pursued to achieve a cure for HIV (Rasmussen et al., 2013). First, intensification studies have explored whether adding an extra antiretroviral drug to an already suppressive cART regimen can reduce the residual viremia or the latent HIV reservoir. Overall, there seems to be little or no effect from these interventions, but there are conflicting results. Elimination of latently infected T cells by reactivating HIV-1 expression using agents like histone deacetylase inhibitors (HDACi), IL-7, disulfiram or prostratin have been investigated in numerous in vitro and in vivo studies. Since reactivation of HIV-1 expression in latently infected cells may be insufficient to ensure the removal of these cells, immunotherapy to enhance HIV specific immunity is continuously being developed and tested.

There are 11 known histone deacetylase (HDAC) metal-dependent enzymes, which are classified into class I (HDAC 1, 2, 3, and 8), class IIa (HDAC 4, 5, 7, and 9), class IIb (HDAC 6 and 10), and class IV (HDAC 11) (Wang et al., Nat. Rev. Drug Discov., 8:969-981 (2009)). The counteracting mechanisms of HDACs and histone acetyl transferases (HAT) exert a key function in regulating gene expression by controlling the degree of acetylation/deacetylation of histone tails, which in turn influences chromatin condensation. The HIV 5′ long-terminal repeat (LTR) that contains promoter and enhancer elements and has binding sites for several transcription factors is arranged in two nucleosomes, nuc-0 and nuc-1. In the transcriptionally silent state of HIV latency, various transcription factors recruit HDACs to the HIV-1 5′ LTR where they induce chromatin condensation by promoting deacetylation of lysine residues on histones, keeping nuc-1 in the hypoacetylated state and preventing HIV transcription. HDAC inhibitors (HDACi) offset these mechanisms by inhibiting HDACs. Chromatin immunoprecipitation assays have shown that the class I HDACs, HDAC1, 2 and 3, may be particularly important to maintaining latency. A recent study correlating HDACi isoform specificity with the ability to reactivate latent HIV-1 expression, showed that potent inhibition or knockdown of HDAC1 was not sufficient to disrupt HIV latency. HDAC3 inhibition was found to be essential for reactivating viral expression. Class I HDACs are ubiquitously expressed and deacetylation of lysine residues on histones is a key function of class I HDACs. However, they may deacetylate more than 1750 non-histone proteins. To which degree, if any, the non-histone effects of HDACi contribute to the desired circumvention of HIV latency is largely unknown.

The HDACi acting on HDAC metalloenzymes may be categorized according to their chemical structure into short chain fatty acids, hydroxamic acids and cyclic tetrapeptides, and are further characterized as selective or pan-inhibitors according to their spectrum of action. Consistent with the role histone deacetylases play in repressing transcription, HDAC inhibitors have been shown to disrupt HIV-latency and induce virus HIV-1 expression in latently infected cell lines, latently infected primary T-cells, resting CD4+T-cells isolated from HIV-infected donors and, recently, in vivo. Valproic acid (VPA), a known anticonvulsant that also exerts weak HDAC inhibition, was the first HDACi to be tested in a clinical study with the objective of depleting the latent reservoir of HIV-1 infection. Whereas a substantial decline was seen in the frequency of replication competent HIV in circulating resting CD4 T cells in the initial study, additional studies failed to demonstrate any effect of VPA, even in the setting of intensified cART.

Vorinostat is a hydroxamic acid containing pan-HDACi with activity against class I and II HDACs. It is the most extensively investigated HDACi in HIV, having consistently shown the ability to reactivate HIV-1 expression at therapeutic concentrations in latently infected cell lines, latently infected primary cells, and resting CD4+ T-cells from HIV infected patients on suppressive HAART. A recent study investigating the HDACi vorinostat, VPA and oxamflatin found that the levels of HIV production by HDAC inhibitor stimulated resting CD4+ T-cells from aviremic donors were not significantly different from those of cells treated with media alone, based on measurement of virion-associated (extracellular) HIV-RNA rather than cell-associated HIV-RNA. Data from a recent clinical trial showed that a single dose of 400 mg Vorinostat significantly increased expression of HIV-RNA in isolated resting CD4 T cells in 8 of 8 evaluated subjects without any safety issues, other than the problematic thrombocytopenia seen with all HDAC inhibitors.

Clinical and experimental studies have identified a range of immune modulatory effects of HDACi involving both specific inflammation signaling pathways (e.g., regulation of NF-κB via IκBα or p65) as well as epigenetic mechanisms. Most of these effects are anti-inflammatory but the biologic roles of individual HDAC isoforms and their corresponding selective inhibitors are complex and show great diversity. HDACi induced immune suppression via Tregs may impact the course of HIV infection since the virus induces excess inflammation that drives disease progression in untreated HIV infection and causes premature immunosenescence and morbidity in persons on HAART. In HIV eradication, the consequences of HDACi induced Treg expansion and/or function, could be either beneficial, by suppressing generalized T-cell activation, or detrimental, by weakening HIV-specific immune responses, thereby hindering immune-mediated clearance of latently infected reactivated CD4 T cells. However, predicting different HDAC is in vivo anti- or pro-inflammatory effects in HIV may prove challenging since even structurally related compounds have been shown to have opposing actions.

Early studies suggested that interleukin (IL)-2 therapy might impact on the frequency of resting cells harboring replication competent virus, but rebound viremia occurred rapidly upon interruption of cART. Additional studies could not establish an effect of IL-2 on the pool of latently infected CD4 T cells or HIV production, and when IL-2 was used in combination with anti-CD3 antibody OKT3 this led to detrimental T cell activation and irreversible CD4 T cell depletion. Several studies have shown that IL-7 induces virus outgrowth ex vivo in the resting CD4 T cells of HIV infected patients on cART (Wang et al., J. Clin. Invest., 115:128-137 (2005); Lehrman et al., J. Acquir. Immune Defic. Syndr., 36:1103-1104 (2004)). Two small clinical trials conducted in HIV infected patients reported that IL-7 administration increased CD4+ and CD8 T cells with a memory phenotype. A recent study showed that, whereas partial reactivation of latent HIV-1 can be achieved with IL-2 and IL-7 in combination, this does not reduce the pool of latently infected cells. Proliferation induced by these cytokines may favor the maintenance of the latent HIV-1 reservoir. Collectively, these findings indicate that the homeostatic proliferation induced by IL-7 therapy could be counterproductive in HIV eradication therapy.

Some toll-like receptor (TLR) ligands appear to modulate latent HIV infection. The TLR-5 agonist flagellin results in NF-κB activation and induces expression in latently infected cell lines and resting central memory T-cells transfected with HIV-1, but could not be shown to reactivate HIV-1 in purified resting CD4 T cells from aviremic HIV patients. The TLR7/8 agonist, R-848, activated HIV from cells of myeloid-monocytic origin through TLR8-mediated NF-κB activation (Schlaepfer et al., J. Immunol., 176:2888-2895 (2006); Schlaepfer and Speck, J. Immunol., 186:4314-4324 (2011)). Finally, synthetic CpG oligodeoxynucleotides (CpG ODNs) that stimulate immune cells via TLR9 induced HIV reactivation in vitro.

In summary, combination ART has transformed HIV from a deadly to a chronic disease, but HIV infected patients are still burdened with excess morbidity and mortality, acquisition of viral resistance to drug regimens, regimen-adherence issues, long-term toxicities from cART, stigmatization and, finally, insufficient access to cART worldwide. A cure for HIV would have a substantial impact on society as well as the individual and continues to be a high research priority.

It is therefore an object of this invention to provide methods and compositions for treatment of HIV infections functionally, to reduce viral load following cessation of drug therapy.

### SUMMARY OF THE INVENTION

Methods and compositions for treatment of human immunodeficiency virus (HIV) infections have been developed which dampen immune activation with a bias more on the CD4 T cells relative to the CD8 T cell response, inhibit HIV replication, reactivate latent HIV, and inhibit infection of cells by HIV. It has been discovered that pushing latent HIV into active infections with inhibition of cell infection by the reactivated HIV can substantially reduce the number of cells infected with HIV and the viral load of HIV, which is not achieved using just the combination of ART and compounds which activate latent HIV. The methods involve administering three or more compounds to an HIV-infected subject collectively dampening immune activation with a bias more on the CD4 T cell relative to the CD8 T cell response, inhibiting HIV replication, reactivating latent HIV, and inhibiting infection of CD4 T cells by HIV, wherein the compounds are provided in dosages substantially reducing the number of cells infected with HIV or the viral load of HIV, relative to which is achieved using just the combination of ART and compounds which activate latent HIV.

Representative inhibitors of HIV replication include nucleoside reverse transcriptase inhibitors (NRTIs) such as tenofovir, emtricitabine, zidovudine (AZT), lamivudine (3TC), abacavir, and tenofovir alafenamide fumarate; non-nucleotide reverse transcriptase inhibitors (NNRTIs) such as efavirenz, rilpivirine, and etravirine; integrase inhibitors such as raltegravir and elvitegravir; and protease inhibitors such as ritonavir, darunavir, atazanavir, lopinavir, and cobicistat. Representative compounds dampening immune activation include anti-inflammatories such as hydroxychloroquine, chloroquine, PD-1 inhibitors, type I interferons, IL6, cyclo-oxygenase-2 inhibitors, peroxisome proliferator-activated receptor-c (PPAR-c) agonists such as pioglitazone and leflunomide, methotrexate, mesalazine, and anti-fibrotic agents such as angiotensin-converting enzyme (ACE) inhibitors. Representative inhibitors of HIV infection of CD4 T cells include C-C chemokine receptor type 5 (CCR5) inhibitors, C-X-X chemokine receptor type 4 (CXCR4) inhibitors, CD4 inhibitors, gp120 inhibitors, and gp41 inhibitors, wherein the stimulator of CD8 T cell response to HIV can be a direct stimulator of CD8 T cell response to HIV, a differential stimulator of CD8 T cell response to HIV, can also be administered. Representative compounds include IL-2, IL-12, IL-15, or a combination thereof, or a composition that stimulates production in the subject of IL-2, IL-12, IL-15, or a combination thereof. Representative compounds that stimulate reactivation of latent HIV include HDACi such as vorinostat, pomidepsin, panpbinostat, givinostat, belinostat, valproic acid, CI-994, MS-275, BML-210, M344, NVP-LAQ824, mocetinostat, and sirtuin inhibitors; NF-κB-inducing agents such as anti-CD3/CD28 antibodies, tumor necrosis factor alpha (TNFα), prostratin, ionomycin, bryostatin-1, and picolog; histone methyltransferase (HMT) inhibitors such as BIX-01294 and chaetocin; pro-apoptotic and cell differentiating molecules such as JQ1, nutlin3, disulfiram, aphidicolin, hexamethylene bisacetamide (HMBA), dactinomycin, aclarubicin, cytarabine, Wnt small molecule inhibitors, Notch inhibitors; immune modulators such as anti-PD-1 antibodies, anti-CTLA-4 antibodies, anti-TRIM-3 antibodies, and BMS-936558; and CD4 T cell vaccines. In the most preferred embodiment, these are administered with a combination of nucleos(t)ide and non-nucleos(t)ide retroviral inhibitors

In preferred embodiments, the inhibitor is a CCR5 inhibitor such as Maraviroc at a dosage of 200 to 600 mg of Maraviroc per day, the the compound dampening immune activation is a chloroquine compound such as hydroxychloroquine in a dosage of between 150 to 400 mg administered per day, the stimulator of reactivation of latent HIV is a histone deacetylase inhibitor such as Vorinostatin a dosage of from 150 to 400 mg administered per day. A clinical study is proposed having the following treatment:

Vorinostat at 400 mg orally every 24 hours for 3 cycles of 14 days with an interim rest-period of 14 days between cycles;

Hydroxychloroquine (H) at a dosage of 200 mg twice daily during the course of vorinostat administration with no rest-period during the interim cycle;

Maraviroc (M) at a dosage of 600 mg twice daily during the course of vorinostat administration with no rest-period during the interim cycle; and

HAART in the form of two-nucleos(t)ide reverse-transcriptase inhibitors such as emtricitabine (FTC) and tenofovir (TDF) and one non-nucleoside reverse transcriptase inhibitor such as efavirenz (EFV) or a protease or integrase inhibitor in subjects who are intolerant to EFV for the duration of the treatment at a dosage equivalent to FTC, 200 mg 1×/day; TDF, 300 mg 1×/day and EFV, 600 mg 1×/day or a protease-inhibitor or integrase-inhibitor.

In one embodiment, the administration of the inhibitors and reactivation stimulator can be a course of treatment including a plurality of administrations of the inhibitors and reactivation stimulator over a period of time. For example, the inhibitors and reactivation stimulator can be administered daily. The period of time can be, for example, from 10 weeks to 40 weeks. In particular embodiments, the period of time can end after the earlier of 40 weeks or 2 weeks after HIV infected cells or HIV viral load becomes undetectable.

In one embodiment, the subject has not been administered any anti-HIV treatment for at least two weeks prior to administration of the inhibitors and reactivation stimulator. In another embodiment, the subject has not been administered any anti-HIV treatment for at least 10 weeks prior to administration of the inhibitors and reactivation stimulator.

In one embodiment, the method include administering to the subject a highly active antiretroviral therapy (HAART), a direct stimulator of CD8 T cell response to HIV and a differential stimulator of CD8 T cell response to HIV. The drugs are preferably administered together, over one or more periods of time. The second period of time can completely overlap with the first period of time, can partially overlap with the first period of time, or can follow the first period of time. In a particular embodiment, no part of the second period of time precedes the first period of time. In a particular embodiment, the second period of time overlaps the last two weeks of the first period of time.

The methods and compositions can result in a CD4 T cell count, HIV viral load and/or HIV infected cell count at or below a threshold level for four weeks, 8 weeks, more preferably 3 months, more preferably 6 months, and most preferably 12 months following the end of a course of treatment. In particular embodiments, the CD4 T cell count can remain at or above 300 per cubic millimeter, preferably 500 per cubic millimeter; HIV viral load can remain at or below 1000 copies per milliliter of blood, preferably 100 copies per milliliter of blood, most preferably undetectable; and/or HIV infected cell count can remain at or below 1% of peripheral blood mononuclear cells, preferably below 0.1% of peripheral blood mononuclear cells, most preferably below 0.01% of peripheral blood mononuclear cells, for 8 weeks, preferably 3 months, more preferably 6 months, and most preferably 12 months following the end of a course of treatment.

### BRIEF DESCRIPTION OF THE DRAWINGS

FIGS. 1A-1H are graphs of the actual results as well as computer modeled simulated results for clinical trials described in the prior art.

FIG. 2 is a graph of HIV virus load (log 10 RNA copies/ml) versus time (weeks) in an immune system simulation of baseline (untreated) HIV infection (upper line at week 52) and treatment holding new infections in check (as with a CCR5 inhibitor), reactivating HIV in latently infected cells (as with a histone deacetylase inhibitor), and stimulation of CD8 T cell response (as with IL-15) (lower line at week 52). The treatment was started at week 26 and continued to week 40.

FIG. 3 is a graph of CD4 T cell count (cells/μl) versus time (weeks) in an immune system simulation of baseline (untreated) HIV infection (lower line at week 52) and treatment holding new infections in check (as with a CCR5 inhibitor), reactivating HIV in latently infected cells (as with a histone deacetylase inhibitor), and stimulation of CD8 T cell response (as with IL-15) (upper line at week 52). The treatment was started at week 26 and continued to week 40.

FIG. 4 is a graph of HIV virus load (log 10 RNA copies/ml) versus time (weeks) in an immune system simulation of baseline (untreated) HIV infection (upper line at week 55) and treatment holding new infections in check (as with a CCR5 inhibitor) and reactivating HIV in latently infected cells (as with a histone deacetylase inhibitor) (lower line at week 55). The treatment was started at week 26 and continued to week 80.

FIG. 5 is a graph of CD4 T cell count (cells/μl) versus time (weeks) in an immune system simulation of baseline (untreated) HIV infection (lower line at week 55) and treatment holding new infections in check (as with a CCR5 inhibitor) and reactivating HIV in latently infected cells (as with a histone deacetylase inhibitor) (upper line at week 55). The treatment was started at week 26 and continued to week 80.

FIG. 6 is a graph of HIV virus load (log 10 RNA copies/ml) versus time (weeks) in an immune system simulation of baseline (untreated) HIV infection (upper line at week 55) and treatment starting at week 26 and ending at week 36 holding new infections in check (as with a CCR5 inhibitor) and reactivating HIV in latently infected cells (as with a histone deacetylase inhibitor), followed by a standard HAART protocol starting at week 34 and ending at week 46 (lower line at week 55).

FIG. 7 is a graph of CD4 T cell count (cells/μl) versus time (weeks) in an immune system simulation of baseline (untreated) HIV infection (lower line at week 55) and treatment starting at week 26 and ending at week 36 holding new infections in check (as with a CCR5 inhibitor) and reactivating HIV in latently infected cells (as with a histone deacetylase inhibitor), followed by a standard HAART protocol starting at week 34 and ending at week 46 (upper line at week 55).

FIG. 8 is a graph of HIV infected cells (log cells) versus time (weeks) in an immune system simulation of treatment inhibiting new infections with Maraviroc and reactivating HIV in latently infected cells with Vorinostat. The lines at week 30, in order from top to bottom, result from increasing effectiveness of Maraviroc at inhibiting new HIV infections. The treatment was started at week 26 and continued to week 78.

FIG. 9 is a graph of HIV infected cells (log cells) versus time (weeks) in an immune system simulation of treatment inhibiting new infections with Maraviroc and hydroxychloroquine and reactivating HIV in latently infected cells with Vorinostat in various combinations. The no treatment base is the only line at 20 weeks, full treatment using effective amounts of all three drugs (VMC) is the lowest line at week 41, and treatment with both Vorinostat and Maraviroc (VM) is the second lowest line at week 41. The other lines at week 104, in order from top to bottom, are treatment with both Vorinostat and Maraviroc (VM), treatment with both hydroxychloroquine alone (C), treatment with both hydroxychloroquine and Maraviroc (MC) and treatment with Maraviron alone (M) (lines overlap), no treatment base, and treatment with both Vorinostat and hydroxychloroquine (VC) and treatment with Vorinostat alone (V) (lines overlap). The treatment was started at week 26 and continued through week 42.

FIG. 10 is a graph of HIV infected cells (log cells) versus time (weeks) in an immune system simulation of treatment inhibiting new infections with Maraviroc and hydroxychloroquine and reactivating HIV in latently infected cells with Vorinostat using varying amounts of Vorinostat. The lines at week 40, in order from top to bottom, are the no treatment base, treatment with hydroxychloroquine, Maraviroc, and Vorinostat at 0.5 (V0.5MC), treatment with hydroxychloroquine, Maraviroc, and Vorinostat at 1 (V1MC), treatment with hydroxychloroquine, Maraviroc, and Vorinostat at 2 (V2MC), treatment with hydroxychloroquine, Maraviroc, and Vorinostat at 4 or 5 (V4MC and V5MC) (lines overlap), and treatment with hydroxychloroquine, Maraviroc, and Vorinostat at 3 (VMC; the full treatment). The treatment was started at week 26 and continued through week 42.

FIG. 11 is a graph of HIV infected cells (log cells) versus time (weeks) in an immune system simulation of treatment inhibiting new infections with Maraviroc and hydroxychloroquine and reactivating HIV in latently infected cells with Vorinostat using varying amounts of Maraviroc. The lines at week 30, in order from top to bottom, are the no treatment base, treatment with hydroxychloroquine, Vorinostat, and Maraviroc at −0.1 (VM0.1C), treatment with hydroxychloroquine, Vorinostat, and Maraviroc at −0.5 (VM0.5C), treatment with hydroxychloroquine, Vorinostat, and Maraviroc at −1.5 (VM1.5C), treatment with hydroxychloroquine, Vorinostat, and Maraviroc at −2 (VM2C; the full treatment), treatment with hydroxychloroquine, Vorinostat, and Maraviroc at −2.5 (VM2.5C), and treatment with hydroxychloroquine, Vorinostat, and Maraviroc at −3 (VM3C). The treatment was started at week 26 and continued through week 42.

FIG. 12 is a graph of HIV infected cells (log cells) versus time (weeks) in an immune system simulation of treatment inhibiting new infections with Maraviroc and hydroxychloroquine and reactivating HIV in latently infected cells with Vorinostat using varying amounts of hydroxychloroquine. The lines at week 35, in order from top to bottom, are the no treatment base, treatment with Maraviroc, Vorinostat, and hydroxychloroquine at −0.01 (VMC0.01), treatment with Maraviroc, Vorinostat, and hydroxychloroquine at −0.05 (VMC0.05), treatment with Maraviroc, Vorinostat, and hydroxychloroquine at −0.1 (VMC; the full treatment), treatment with Maraviroc, Vorinostat, and hydroxychloroquine at −0.2 (VMC0.2), treatment with Maraviroc, Vorinostat, and hydroxychloroquine at −0.4 (VMC0.4), and treatment with Maraviroc, Vorinostat, and hydroxychloroquine at −0.6 (VMC0.6). The treatment was started at week 26 and continued through week 42.

### DETAILED DESCRIPTION OF THE INVENTION

Methods and compositions for treatment of human immunodeficiency virus (HIV) infections have been developed. Efforts to cure individuals of HIV infection have been stymied by a remaining reservoir of latently infected T cells. Front line anti-HIV treatments generally target only active HIV infections and cannot reach cells that are latently infected. If anti-HIV treatment is paused or stopped, reactivation of latent HIV can generate newly infected cells and resurgent viral loads. Lifelong treatment with anti-HIV therapy has been the only answer to this problem.

Latent HIV infection must be attacked to produce more robust and longer lasting reduction in infected cell counts and viral load. The approach disclosed herein involves reactivation of latent HIV and inhibiting infection of cells by HIV, in combination with inhibitors of viral repliaction. A combination of driving HIV out of latency with inhibition of cell infection and viral replication by the reactivated HIV substantially reduces the number of cells infected with HIV and the viral load of HIV.

Some factors can affect the effectiveness of the methods and compositions. Because HIV targets the immune system, the state of the immune system can affect reactivation of latent HIV, cell infection by HIV, and HIV replication. Having a more active immune response can increase the effectiveness of the methods. It is believed that a more active cellular cytotoxic response leads to more effective hindrance of cell infection by HIV. For this reason, and because anti-HIV therapy (such as standard HAART) may result in a waning of the measurable CD8 T cell immune response, it may be useful for subjects to be treatment-experienced at an early stage of HIV infection (within 12 months) or treatment-naïve but at an early stage of HIV infection (within 3 months) when the cellular immune response is more intact before treatment with the methods and compositions disclosed herein.

The method uses inhibitors of HIV infection that have different effects or targets of action. For example, it has been discovered that a combination of the CCR5 inhibitor Maraviroc, which inhibits HIV entry into cells via the CCR5 receptor, thus slowing infection of CD4 T cells, and hydroxychloroquine, which reduces viral replication by reducing the inflammatory response that accompanies HIV infection, improves the effectiveness of inhibition of HIV infection of CD4 T cells. Hydroxychloroquine has been shown in HIV treatment trials to have less of an impact on CD8 T cell function relative to its impact on CD4 T cell function (Piconi et al., Blood, 118(12):3263-72 (2011)). The method can be made more effective by using one or more different inhibitors of HIV infection, preferably having different effects or targets of action, and/or by using one or more stimulators of reactivation of latent HIV, preferably having different effects or targets of action. Preferably, HAART is included to further hinder cell infection by HIV and HIV replication, thus helping to reduce the viral load and HIV infected cell count. As another example, CD8 T cell response to HIV can be stimulated and/or differentially regulated relative to CD4 T cell responses in blood in the method. The immune system's attack on HIV infected cells can thus help to decrease the viral load and HIV infected cell count once or as latent HIV is reactivated by the method.

### I. DEFINITIONS

As used herein, “active infection” and “active viral infection” refer to a viral infection where viral replication and production is ongoing. Production of virus refers to production of copies of viral genomes and production of viral particles. Unless noted otherwise, all references herein to “HIV” refer to HIV-1 and all genomic subtypes within HIV-1.

A “plurality of administrations” refers to multiple administrations made at different times, different routes, and/or different forms. In the context of a plurality of administrations over a period of time, the plurality of administrations at least refers to multiple administrations made at different times during the period of time.

As used herein, “anti-HIV therapy” refers to a treatment or therapy that has the purpose of reducing the number of cells infected with HIV, reducing HIV viral load, or both.

As used herein, “anti-HIV therapy holiday” refers to a break or pause in administration of anti-HIV therapies to a subject. As used herein, a subject that “has not been administered any anti-HIV treatment” refers to subjects that are naïve to anti-HIV therapy or that are on an anti-HIV therapy holiday. The latter is generally used in the context of a subject that has not been administered any anti-HIV treatment for a specified period of time.

As used herein, “cell count” refers to the number of cells having a specified characteristic. For example, an HIV infected cell count refers to the number of cells infected with HIV. A CD4 T cell count refers to the number of CD4 T cells. Cell count is generally based on or expressed relative to a volume or amount of sample tested. Thus, for example, a direct or derived measurement of 10 HIV infected cells in a 5 μl sample of blood can be expressed as a cell count of 2/n1 of blood, 2,000/ml, or some other equivalent. As used herein, expressions such as “HIV infected cells are no longer detected” and “HIV infected cells are undetectable” refer to HIV infected cell counts that are undetectable under the assay conditions used.

As used herein, “course of treatment” refers to a plurality of administrations that follow a plan or schedule of treatment.

As used herein, “effective amount” of a compound or composition refers therapeutically effective amount of the compound to provide the desired result.

As used herein, “following” refers to an event or act that takes place after a period of time, existence of a condition, or a prior act or event has ended or no longer exists. For example, administering HAART following a course of treatment with a stimulator of reactivation of latent HIV means that the HAART is administered after the course of treatment with the stimulator has ended.

As used herein, “precedes” refers to an event or act that takes place before a period of time, existence of a condition, or a prior act or event has begun or no exists. For example, administration of a stimulator of reactivation of latent HIV preceding HAART means that the stimulator is administered before the HAART treatment.

As used herein, “virus infection of a cell” refers to entry of virus into a cell and the beginning of an active infection of the cell. Unless the context indicates otherwise, this is meant to refer to the event of the virus beginning infection of a cell. Ongoing viral infections can be referred to as active viral infections. Active viral infections generate new events of viral infection of cells. “HIV infection of T cells” refers to entry of HIV into T cells and the beginning of an active infection of the T cells. Unless the context indicates otherwise, this is meant to refer to the event of HIV beginning infection of a T cell. Ongoing HIV infections can be referred to as active HIV infections. Active HIV infections generate new events of HIV infection of T cells.

As used herein, “inhibiting” refers to reduction or decrease in activity or expression. For example, inhibiting HIV infection of T cells refers to a reduction or decrease in entry of HIV into T cells and the beginning of an active infection of the T cells compared to a control or standard level. This can be a complete inhibition or activity or expression, or a partial inhibition. Inhibition can be compared to a control or to a standard level.

As used herein, “inhibitor of cell infection by virus” refers to a compound or composition that inhibits virus infection of a cell. For example, inhibitor of cell infection by HIV refers to a compound or composition that inhibits HIV infection of a cell.

As used herein, “inhibitor of viral production” refers to a compound or composition that inhibits production of virus. For example, inhibitor of HIV production or replication refers to a compound or composition that inhibits production of HIV.

As used herein, “latent viral infection” refers to a viral infection where the viral genome is incorporated into a chromosome (as a provirus) and is dormant and there is not an active infection. Latent viral infection can refer to a subject as a whole or, more commonly, to cells. Thus, for example, a cell of a subject can be latently infected while other cells in the subject can be actively infected. Latent HIV infection refers to an HIV infection where the HIV genome is incorporated into a chromosome (as a provirus) and is dormant and there is not an active infection.

As used herein, “overlapping with” refers to an event or act that takes place during a specified period of time, during the existence of a condition, or while an act or event is ongoing or exists. For example, a first period of time can be overlapping with a second period of time. For example, a course of treatment of HAART administered during a first period of time overlaps with a course of treatment with a stimulator of reactivation of latent HIV during a second period of time when the first and second periods of time overlap. Put another way, a course of treatment of HAART overlaps with a course of treatment with a stimulator of reactivation of latent HIV when any administrations in the course of HAART treatment are at the same time as or interspersed with administrations of the course of stimulator treatment.

As used herein, “completely overlaps with” refers to an event or act that takes place completely and only during a specified period of time, during the existence of a condition, or while an act or event is ongoing or exists. That is, no part of the event or act takes place outside of, before, or after the specified period of time, the existence of the condition, or the other act or event. For example, a first period of time completely overlaps with a second period of time when no part of the first period of time is outside of the second period of time.

As used herein, “partially overlaps with” refers to an event or act that takes place partially during a specified period of time, during the existence of a condition, or while an act or event is ongoing or exists and partially outside of, before, or after the specified period of time, the existence of the condition, or the other act or event. For example, a first period of time partially overlaps with a second period of time when part of the first period of time overlaps with the second period of time and part of the first period of time is outside of the second period of time. As used herein, “partially overlaps and follows” refers to an event or act that takes place partially during a specified period of time, during the existence of a condition, or while an act or event is ongoing or exists and partially after the specified period of time, the existence of the condition, or the other act or event. For example, a first period of time partially overlaps and follows a second period of time when part of the first period of time overlaps with the second period of time and part of the first period of time is after the second period of time. Similarly, a first period of time partially overlaps and precedes a second period of time when part of the first period of time overlaps with the second period of time and part of the first period of time is before the second period of time.

As used herein, “period of time” refers to a specified continuous interval of time. As used herein, “no part of a period of time” refers to a period of time, event, or act that does not overlap with the specified period of time. As used herein, “sequential time periods” refers to periods of time that follow one another. Unless otherwise noted, the sequential time periods do not overlap. There may or may not be gaps in time between the sequential time periods.

As used herein, “pharmaceutically acceptable” refers to a material that is not biologically or otherwise undesirable; that is, the material can be administered to a subject along with the selected compound without causing undesirable biological effects or interacting in a deleterious manner with the other components of the pharmaceutical composition in which it is contained.

As used herein, “reactivation” refers to a shift of a provirus from latency or dormancy into active infection.

As used herein, “reduce” refers to decrease in number, amount, or level. For example, reducing HIV viral load refers to a reduction or decrease in the amount of HIV in an involved body fluid. Reduction generally can be compared to an initial of starting number, amount, or level, but can also be compared to a control or to a standard number, amount, or level.

As used herein, “selectively affects” refers to a compound, composition, treatment, condition, etc. that has a greater effect on one component or condition as compared to another component or condition. For example, in the context of immune responses, a composition can be said to selectively affect, for example, CD4 T cell-based immune response as compared to CD8 T cell-based immune response. For example, an anti-inflammatory compound can selectively affect CD4 T cells compared to CD8 T cells, meaning, for example, that the CD4 T cell immune response is inhibited while the CD8 T cell immune response is not inhibited or is less inhibited than the CD4 T cell immune response.

As used herein, “separate administration” refers to an administration that is of a separate composition, at a different time, by a different route, and/or in a different manner than another administration.

As used herein, “separate composition” refers to a composition that is physically separate from another composition. For example, different pills that are not bound or attached to each other are separate compositions. As another example, two liquid solutions that are mixed together are not separate compositions once they are mixed together.

As used herein, “single composition” refers to a combination of components in one composition rather than in separate compositions. For example, a first inhibitor of HIV infection of CD4 T cells, a second inhibitor of HIV infection of CD4 T cells, and a stimulator of reactivation of latent HIV formulated in a single pill are in a single composition.

As used herein, “stimulator of reactivation of the latent virus” refers to a compound or composition that stimulates or promotes a shift of a provirus from latency or dormancy into active infection. For example, stimulator of reactivation of the latent HIV refers to a compound or composition that stimulates a shift of an HIV provirus from latency or dormancy into active HIV infection.

As used herein, “stimulator of CD8 T cell response to HIV” refers to a compound or composition that stimulates, increases, or promotes a CD8 T cell response to HIV. Such stimulation can be relative to a prior or baseline CD8 T cell response to HIV (this can be referred to as direct stimulation of CD8 T cell response to HIV) and/or such stimulation can be relative to CD4+ activation (this can be referred to as differential stimulation of CD8 T cell response to HIV). For example, a stimulator of CD8 T cell response to HIV can increase CD8 T cell response to HIV relative to the prior existing CD8 T cell response to HIV, can decrease CD4 T cell activation with no or a lesser decrease of the prior existing CD8 T cell response to HIV, or can both increase CD8 T cell response to HIV relative to the prior existing CD8 T cell response to HIV and decrease CD4 T cell activation.

A “direct stimulator” of CD8 T cell response to HIV supports direct stimulation of CD8 T cell response to HIV. A “differential stimulator” of CD8 T cell response to HIV supports direct stimulation of CD8 T cell response to HIV. Generally, an increase of CD8 T cell response to HIV relative to the prior existing CD8 T cell response to HIV can be accomplished with a direct stimulator of CD8 T cell response to HIV. Generally, a decrease of CD4 T cell activation with no or a lesser decrease of the prior existing CD8 T cell response to HIV can be accomplished with a differential stimulator of CD8 T cell response to HIV. Generally, a combination of an increase of CD8 T cell response to HIV relative to the prior existing CD8 T cell response to HIV and a decrease of CD4 T cell activation can be accomplished with a direct stimulator and a differential stimulator of CD8 T cell response to HIV.

As used herein, “subject” refers to a human.

As used herein, “viral load” refers to the amount of virus in an involved body fluid. For example, viral load can be given in viral copies per milliliter of blood plasma. HIV viral load refers to the amount of HIV in an involved body fluid. Viral load is a measure of the severity of a viral infection. Tracking viral load is used to monitor therapy during chronic viral infections. As used herein, “HIV viral load becomes undetectable” refers to the condition where no virus is detected in the sample being tested by standard commercial quantitative viral load assays. Because of limits of assay methods, HIV can be undetectable in an assay when virus is still present in the sample, albeit at a very low level. HIV is considered to be functionally absent when HIV viral load is undetectable.

### II. COMPOSITIONS

Inhibitors of HIV Infection of CD4 T Cells

Compounds that inhibit HIV infection of CD4 T cells include, for example, entry inhibitors, such as C-C chemokine receptor type 5 (CCR5) inhibitors, C-X-X chemokine receptor type 4 (CXCR4) inhibitors, CD4 inhibitors, gp120 inhibitors, and gp41 inhibitors (such as enfuvirtide); and anti-inflammatories, such as hydroxychloroquine, chloroquine, PD-1 inhibitors, type I interferons, IL6, cyclo-oxygenase-2 inhibitors, peroxisome proliferator-activated receptor-c (PPAR-c) agonists (such as pioglitazone and leflunomide), methotrexate, mesalazine, and anti-fibrotic agents (such as angiotensin-converting enzyme (ACE) inhibitors). Examples of CCR5 inhibitors include maraviroc, aplaviroc, and vicriviroc. Examples of other entry inhibitors include TNX-355, PRO 140, BMS-488043, plerixafor, epigallocatechin gallate, anti-gp120 antibody, such as antibody b12, griffithsin, DCM205, and Designed Ankyrin Repeat Proteins (DARPins).

Maraviroc (Pfizer) is an antiretroviral drug in the CCR5 receptor antagonist class used in the treatment of HIV infection. It is also classed as an entry inhibitor. It also appeared to reduce graft-versus-host disease in patients treated with allogeneic bone marrow transplantation for leukemia. Maraviroc is a virus entry inhibitor. Specifically, Maraviroc is a negative allosteric modulator of the CCR5 receptor. The drug binds to CCR5, thereby blocking the HIV protein gp120 from associating with the receptor. HIV is then unable to enter human macrophages and T-cells. Because HIV can also use other co-receptors, such as CXCR4, an HIV tropism test such as a Trofile assay should be performed to determine if the drug will be effective. Maraviroc is administered twice daily, at a dosage of 600 mg daily when co-administered with certain antiretroviral medicals, 300 mg daily when administered with CYP3A inhibitors such as a protease inhibitor like tipranavir or delavirdine, or 1200 mg daily when administered with a CYP3A inducer such as efavirenz or etravirine.

Chloroquine is a 4-aminoquinoline drug used in the treatment or prevention of malaria. Chloroquine was discovered in 1934 and clinical trials for antimalarial drug development during World War II showed that chloroquine has a significant therapeutic value as an antimalarial drug. It was introduced into clinical practice in 1947 for the prophylactic treatment of malaria. Chloroquine inhibits thiamine uptake. It acts specifically on the transporter SLC19A3. As an antiviral agent, chloroquine impedes the completion of the viral life cycle by inhibiting some processes that occur within intracellular organelles and that require a low pH. As for HIV-1, chloroquine inhibits the glycosylation of the viral envelope glycoprotein gp120, which occurs within the Golgi apparatus.

Hydroxychloroquine is also an antimalarial drug and is used to reduce inflammation in the treatment of rheumatoid arthritis and lupus. Hydroxychloroquine differs from chloroquine by the presence of a hydroxyl group at the end of the side chain: The N-ethyl substituent is beta-hydroxylated. It is available for oral administration as hydroxychloroquine sulfate (PLAQUENIL) of which 200 mg contains 155 mg base in chiral form. Hydroxychloroquine has similar pharmacokinetics to chloroquine, with quick gastrointestinal absorption and is eliminated by the kidney. Cytochrome P450 enzymes (CYP 2D6, 2C8, 3A4 and 3A5) converts N-desethylated hydroxychloroquine to N-desethylhydroxychloroquine.

Hydroxychloroquine is used to treat systemic lupus erythematosus, rheumatic disorders like rheumatoid arthritis and Sjögren's Syndrome, and porphyria cutanea tarda. Hydroxychloroquine increases lysosomal pH in antigen presenting cells. In inflammatory conditions, it blocks TLR on plasmacytoid dendritic cells (pDCs). TLR 9, which recognizes DNA-containing immune complexes, leads to the production of interferon and causes the dendritic cells to mature and present antigen to T cells. Hydroxychloroquine, by decreasing TLR signaling, reduces the activation of dendritic cells and the inflammatory process.

Hydroxychloroquine and its quinoline analogue chloroquine have been used in HIV-1 therapeutic trials since 1995 (Sperber et al., Clin. Ther. 1995 July-August; 17(4):622-36.). Both drugs are similar in structure with identical biological mechanisms. The free base form of the drugs accumulates in lysosomes, increasing the pH to levels that inhibit lysosomal proteases, thereby diminishing intracellular processing, glycosylation, and secretion of cellular proteins. These drugs interfere with a number of steps in the T-cell activation pathway including antigen-presentation (Ziegler and Unanue, Proc. Natl. Acad. Sci. U.S.A. 1982 January; 79(1):175-8), T-cell receptor-mediated intracellular calcium signaling (Goldman et al., Blood. 2000 Jun. 1; 95(11):3460-6), the reduction of pro-inflammatory cytokine production (Sperber et al., J. Rheumatol., 1993 May; 20(5):803-8) and modulation of the intracellular TLR pathway (Hong et al., Int. Immunopharmacol., 2004 February; 4(2):223-34). Additionally, hydroxychloroquine and chloroquine have antiviral properties resulting in inhibition of viral protein glycosylation (Savarino et al., J. Acquir. Immune Defic. Syndr., 2004 Mar. 1; 35(3):223-32).

The use of hydroxychloroquine and chloroquine in HIV therapeutic trials has been either singly or in combination with anti-retroviral therapy (Paton et al., JAMA., 2012 Jul. 25; 308(4):353-61; Piconi et al., Blood, 2011 Sep. 22; 118(12):3263-72). However, the effect of hydroxychloroquine appears to be more significant on CD4+ compared to CD8 T cells in terms of dampening immune activation, with a significant effect on the former, but minimal impact on the latter (Piconi et al., Blood, 2011). Such selective effect on CD4+ and CD8 T cells is useful because a reduction in activation of CD4 T cell-based immune response aids in inhibiting HIV infection of CD4 T cells while CD8 T cell-based immune response aids in clearing HIV infected cells. Thus, it is preferred that the anti-inflammatory compound selectively affects CD4 T cells versus CD8 T cells.

AMD070 (Genzyme) is an entry inhibitor specific for CXCR4. AMD-070 is a selective, reversible, small molecule CXCR4 chemokine coreceptor antagonist. AMD-070 prevents CXCR4-mediated viral entry of T-cell tropic synctium-inducing HIV (associated with advanced stages of HIV-1 infection) by binding to transmembrane regions of the coreceptor, blocking the interaction of the CD4-gp120 complex with the ECL2 domain of the CXCR4 coreceptor. AMD-070 is administered orally and twice daily in 200 mg doses. In healthy participants, the median estimated terminal half-life ranged from 7.6 to 12.6 hours (single-dose cohorts, 50 to 400 mg) and from 11.2 to 15.9 hours (multiple-dose cohorts, 100 to 400 mg twice daily).

Aplaviroc (INN, GW873140) (GlaxoSmithKline) is a CCR5 entry inhibitor developed for the treatment of HIV infection. Aplaviroc is administered orally at 100 mg twice daily, 200 mg twice daily or 400 mg once daily.

BMS-488043 (Bristol Meyers-Squibb) is a unique oral small-molecule inhibitor of the attachment of human immunodeficiency virus type 1 (HIV-1) to CD4⁺ lymphocytes. BMS-488043 is administered orally at 800 mg or 1,800 mg twice daily.

BMS-663068 (Bristol Meyers-Squibb) is a HIV-1 entry inhibitor. BMS-663068 is a methyl phosphate prodrug of the small molecule inhibitor BMS-626529. BMS-626529 prevents viral entry by binding to the viral envelope gp120 and interfering with virus attachment to the host CD4 receptor. BMS-663068 is administered orally in various doses and dosing schedules with total daily BMS-663068 doses ranging from 1200 mg to 2400 mg. For example, 400 or 800 mg twice daily; or 600 or 1200 mg once daily.

Cenicriviroc (TBR-652, CVC, TAK-652) (Takeda; Tobira Therapeutics) is a HIV-1 entry inhibitor. Cenicriviroc is a small-molecule CCR5 coreceptor antagonist that prevents viral entry by binding to a domain of CCR5 and subsequently inhibiting the interaction between HIV-1 gp120 and CCR5. Cenicriviroc is also a CCR2 antagonist. Cenicriviroc is administered once daily and orally. Cenicriviroc doses range from 25 mg to 150 mg.

DCM205 is a small molecule based on L-chicoric acid, an integrase inhibitor. DCM205 is an entry inhibitor specific for CCR5 and CXCR4.

Dolutegravir (DTG, GSK1349572, S/GSK1349572) (ViiV Healthcare) is a HIV-1 integrase strand transfer inhibitor. Dolutegravir prevents viral DNA integration into the host genome. Dolutegravir tablets are administered orally and without regard to food at a dose of 50 mg once or twice daily.

Enfuvirtide (T20) (Roche) is a fusion inhibitor (interferes with gp41 fusion to the cell membrane). Enfuvirtide is administered subcutaneously at 90 mg twice daily.

Epigallocatechin gallate (EGCG), also known as epigallocatechin-3-gallate, is the ester of epigallocatechin and gallic acid, and is a type of catechin. EGCG is the most abundant catechin in tea and is a potent antioxidant that may have therapeutic applications in the treatment of many disorders (e.g. cancer). It is found in green tea, but not black tea. EGCG is administered orally once daily at 800 mg.

Griffithsin is an entry inhibitor specific for CCR5 and CXCR4.

Ibalizumab (Hu5A8, TMB-355. TNX-355) (TaiMed Biologics) is an entry inhibitor specific for CCR5/CXCR4. Ibalizumab allows binding to CD4 but interferes with co-receptor binding. Ibalizumab, a humanized monoclonal antibody (mAb), binds to extracellular domain 2 of the CD4 receptor. The ibalizumab binding epitope is located at the interface between domains 1 and 2, opposite from the binding site for major histocompatibility complex class II molecules and gp120 attachment. Ibalizumab's post-binding conformational effects prevent viral entry and fusion. Ibalizumab can be administered via IV infusion at a dose of 10 mg/kg weekly, 15 mg/kg biweekly, 800 mg every 2 weeks, or 2000 mg every 4 weeks.

INCB-9471 (INCB009471) (Incyte) is a HIV-1 entry inhibitor. INCB-9471 is a selective, reversible, small-molecule CCR5 coreceptor antagonist that binds to a CCR5 binding pocket that is different from what Maraviroc binds to. INCB-9471 prevents viral entry by inhibiting the interaction between HIV-1 gp120 and CCR5. INCB-9471 prevents CCR5-mediated viral entry via allosteric noncompetitive mechanisms. INCB-9471 does not inhibit CXCR4-tropic or dual-tropic viruses. INCB-9471 is administered once daily in a dose of 100 mg or 200 mg of an immediate-release formulation or 300 mg of a slow-release formulation.

Plerixafor (AMD3100) (Genzyme) is an entry inhibitor specific for CXCR4. It is administered in a dosage of 0.16 to 0.24 mg/kg for cancer therapy.

PRO 140 (PA14) (CytoDyn Inc) is a HIV-1 entry inhibitor. PRO-140, a humanized IgG4 monoclonal antibody (mAb), binds to hydrophilic extracellular domains on CCR5, and via competitive mechanisms it inhibits CCR5-mediated HIV-1 viral entry, without preventing CC-chemokine signaling at antiviral concentrations. PRO-140 does not inhibit CXCR4-using viruses. PRO-140 can be administered via SC or IV infusion at a dose of 5 mg/kg or 10 mg/kg.

Sifuvirtide is a fusion inhibitor (interferes with gp41 fusion to the cell membrane).

Vicriviroc is an entry inhibitor specific for CCR5. It is administered in a dosage of 20-30 mg/day. Caseiro, et al. J Infect. 2012 October; 65(4):326-35.

Inhibitors of HIV Production

Compounds that inhibit production of HIV include nucleoside reverse transcriptase inhibitors (NRTIs), such as tenofovir, emtricitabine, zidovudine (AZT), lamivudine (3TC), abacavir, and tenofovir alafenamide fumarate; and one or more non-nucleotide reverse transcriptase inhibitors (NNRTIs), such as efavirenz, rilpivirine, and etravirine; integrase inhibitors, such as raltegravir and elvitegravir; and/or protease inhibitors, such as ritonavir, darunavir, atazanavir, lopinavir, and cobicistat.

HAART is used to reduce the likelihood of the virus developing resistance. The WHO has recently recommended that HAART be initiated when the CD4 T cell count declines to 500 or less/ul (IAS Conference, Kuala Lumpur, Malaysia, 2013). Data suggest that these recommendations mean a substantial increase in the number of patients who will require treatment and need early HIV testing. Six classes of antiretroviral agents currently exist, as follows: nucleoside reverse transcriptase inhibitors (NRTIs), nonnucleoside reverse transcriptase inhibitors (NNRTIs), protease inhibitors (PIs), integrase inhibitors (Hs), fusion inhibitors (FIs), chemokine receptor antagonists (CRAs).

Each class targets a different step in the viral life cycle as the virus infects a CD4⁺ T lymphocyte or other target cell. The use of these agents in clinical practice is largely dictated by their ease or complexity of use, side-effect profile, efficacy based on clinical evidence, practice guidelines, and clinician preference. Resistance, adverse effects, pregnancy, and coinfection with hepatitis B virus, or hepatitis C virus present important challenges to clinicians when selecting and maintaining therapy.

Compounds for HAART are well known and include, for example, a combination of two or more nucleoside reverse transcriptase inhibitors (NRTIs), such as tenofovir, emtricitabine, zidovudine (AZT), lamivudine (3TC), abacavir, and tenofovir alafenamide fumarate; and one or more non-nucleotide reverse transcriptase inhibitors (NNRTIs), such as efavirenz, rilpivirine, and etravirine; integrase inhibitors, such as raltegravir and elvitegravir; and/or protease inhibitors, such as ritonavir, darunavir, atazanavir, lopinavir, and cobicistat. HAART medicines that are most often used to treat HIV infection include nucleoside/nucleotide reverse transcriptase inhibitors, such as tenofovir, emtricitabine, and abacavir; and non-nucleoside reverse transcriptase inhibitors (NNRTIs), such as efavirenz, nevirapine, or etravirine; protease inhibitors (PIs), such as atazanavir, ritonavir, or darunavir; fusion and entry inhibitors, such as enfuvirtide and maraviroc; and integrase inhibitors, such as raltegravir.

Abacavir (ZIAGEN) is a carbocyclic synthetic nucleoside analogue. Abacavir is converted by cellular enzymes to the active metabolite, carbovir triphosphate (CBV-TP), an analogue of deoxyguanosine-5′-triphosphate (dGTP). CBV-TP inhibits the activity of HIV-1 reverse transcriptase (RT) both by competing with the natural substrate dGTP and by its incorporation into viral DNA. The lack of a 3′-OH group in the incorporated nucleotide analogue prevents the formation of the 5′ to 3′ phosphodiester linkage essential for DNA chain elongation, and therefore, the viral DNA growth is terminated. CBV-TP is a weak inhibitor of cellular DNA polymerases α, ρ, and γ. The recommended oral dose of abacavir (ZIAGEN) for adults is 600 mg daily, administered as either 300 mg twice daily or 600 mg once daily, in combination with other antiretroviral agents.

ATRIPLA is a combination of Efavirenz 600 mg, emtricitabine 200 mg, and tenofovir disoproxil fumarate 300 mg.

COMBIVIR (GlaxoSmithKline) is a combination of zidovudine 300 mg+lamivudine 150 mg. COMBIVIR is administered orally twice daily. COMPLERA (Gilead) is a combination of emtricitabine 200 mg+rilpivirine 25 mg+tenofovir 300 mg. COMPLERA is administered orally daily.

Darunavir (PREZISTA) is a second-generation protease inhibitor (PI). Darunavir is administered orally at 600 mg twice a day or 800 mg four times a day.

Didanosine (VIDEX, Didex) (Bristol-Myers Squibb) is a nucleoside reverse transcriptase inhibitor. Didanosine given orally: Patient weight <60 kg: (Tablets): 125 mg orally twice daily or 250 mg once daily or 167 mg (Buffered powder) twice daily. Patient weight >60 kg: (Tablets): 200 mg orally twice daily or 400 mg orally once daily. (Buffered Powder): 250 mg orally twice daily.

Emtricitabine, a synthetic nucleoside analog of cytidine, is phosphorylated by cellular enzymes to form emtricitabine 5′-triphosphate. Emtricitabine 5′-triphosphate inhibits the activity of the HIV-1 reverse transcriptase by competing with the natural substrate deoxycytidine 5′-triphosphate and by being incorporated into nascent viral DNA which results in chain termination. Emtricitabine 5′-triphosphate is a weak inhibitor of mammalian DNA polymerase α, β, ε, and mitochondrial DNA polymerase γ. The dose for adults is 200 mg orally once daily.

Epzicom is a combination of abacavir 600 mg+lamivudine 300 mg. Epzicom is administered orally once daily.

Lamivudine (3TC) is a synthetic nucleoside analogue. Intracellularly lamivudine is phosphorylated to its active 5′-triphosphate metabolite, lamivudine triphosphate (3TC-TP). The principal mode of action of 3TC-TP is inhibition of RT via DNA chain termination after incorporation of the nucleotide analogue. CBV-TP and 3TC-TP are weak inhibitors of cellular DNA polymerases α, β, and γ. The adult dose is one tablet (abacavir 600 mg and lamivudine 300 mg) once daily.

Etravirine is a non-nucleoside reverse transcriptase inhibitor. Etravirine is administered orally twice daily at 200 mg.

Stavudine (ZERIT) is given to patients weight more than 60 kg at a dose of 40 mg orally twice daily; at a dose of 30 mg orally twice daily for patients weighing less than 60 kg. Tenofovir (VIREAD) is given at a dose of 300 mg orally once daily with a meal. TRIZAVIR is a combination of Abacavir 300 mg, lamivudine 150 mg, and zidovudine 300 mg. TRUVADA is a combination of emtricitabine 200 mg and tenofovir 300 mg. Zalcitabine (HIVID) is administered as 0.75 mg orally three times daily. Zidovudine (RETROVIR) is given orally at a dose of 300 mg twice daily or 200 mg 3 times/day.

Atazanavir (Reyataz) (Bristol Myers-Squibb) is a protease inhibitor. Atazanavir is administered orally at 300 mg or 400 mg once daily.

Cobicistat (GS-9350) (Gilead) is a booster of protease inhibitors that inhibits cytochrome P450. Cobicistat is administered daily orally at 150 mg.

Efavirenz (SUSTIVA) (Bristol-Myers Squibb) is a non-nucleoside reverse transcriptase inhibitor. Efavirenz is administered orally at 300 or 600 mg once daily.

Elviegravir (EVG, GS-9137, K-303) (Japan Tobacco Inc.; Gilead Sciences; GlaxoSmithKline) is a HIV-1 integrase strand transfer inhibitor. Elvitegravir prevents viral DNA integration into the host genome. Elvitegravir is administered orally and once daily in combination with a boosting agent (CYP3A inhibitor) and with food at a dose at 85 mg or 150 mg.

S/GSK1265744 (GSK-1265744, GSK1265744, S-265744) (ViiV Healthcare) is a HIV-1 integrase strand transfer inhibitor. S/GSK1265744 prevents viral DNA integration into the host genome. S/GSK1265744 LAP can be administered via IM or SC injection; 800-mg loading dose given at Month 1, followed by monthly maintenance doses (200 mg or 400 mg). S/GSK1265744 can be administered once daily and orally at a dose at 10, 30, or 60 mg.

The U.S. National Institutes of Health recommends one of the following programs for people who begin treatment for HIV:

Efavirenz+tenofovir+emtricitabine;

Ritonavir-boosted atazanavir+tenofovir+emtricitabine;

Ritonavir-boosted darunavir+tenofovir+emtricitabine;

Raltegravir+tenofovir+emtricitabine.

Fixed dose combinations are multiple antiretroviral drugs combined into a single pill:

COMBIVIR: zidovudine and lamivudine; TRIZIVIR: abacavir, zidovudine and lamivudine; KALETRA: lopinavir and ritonavir: EPZICOM: abacavir and lamivudine; TRUVADA: tenofovir and emtricitabine; ATRIPLA: efavirenz, tenofovir and emtricitabine; COMPLERA: rilpivirine, tenofovir, and emtricitabine; and STRIBILD: elvitegravir, cobicistat, tenofovir and emtricitabine.

The preferred initial regimens in the United States are: tenofovir/emtricitabine (a combination of two NRTIs) and efavirenz (a NNRTI); tenofovir/emtricitabine and raltegravir (an integrase inhibitor); tenofovir/emtricitabine, ritonavir, and darunavir (both latter are protease inhibitors); tenofovir/emtricitabine, ritonavir, and atazanavir (both latter are protease inhibitors). Most current HAART regimens consist of three drugs: 2 NRTIs+a PI/NNRTI/II. Initial regimens use “first-line” drugs with a high efficacy and low side-effect profile.

Stimulators of Reactivation of Latent HIV

Compounds that stimulate reactivation of latent HIV include, for example, histone deacetylase (HDAC) inhibitors, such as vorinostat, pomidepsin, panobinostat, givinostat, belinostat, valproic acid, CI-994, MS-275, BML-210, M344, NVP-LAQ824, mocetinostat, and sirtuin inhibitors; NF-κB-inducing agents, such as anti-CD3/CD28 antibodies, tumor necrosis factor alpha (TNFα), prostratin, ionomycin, bryostatin-1, and picolog; histone methyltransferase (HMT) inhibitors, such as BIX-01294 and chaetocin; pro-apoptotic and cell differentiating molecules, such as JQ1, nutlin3, disulfiram, aphidicolin, hexamethylene bisacetamide (HMBA), dactinomycin, aclarubicin, cytarabine, Wnt small molecule inhibitors, and Notch inhibitors; immune modulators, such as anti-PD-1 antibodies, anti-CTLA-4 antibodies, anti-TRIM-3 antibodies, and BMS-936558; and CD4 T cell vaccines. Combinations of such stimulators can also be used. The effects of some stimulators on reactivation of HIV can also be enhanced by combination with other compounds.

Histone deacetylase inhibitors (HDAC inhibitors, HDACi) are a class of compounds that interfere with the function of histone deacetylase. HDAC inhibitors have a long history of use in psychiatry and neurology as mood stabilizers and anti-epileptics. More recently they have been investigated as treatments for cancers and inflammatory diseases. To carry out gene expression, a cell must control the coiling and uncoiling of DNA around histones. This is accomplished with the assistance of histone acetylases (HAT), which acetylate the lysine residues in core histones leading to a less compact and more transcriptionally active chromatin, and, on the converse, the actions of histone deacetylases, which remove the acetyl groups from the lysine residues leading to the formation of a condensed and transcriptionally silenced chromatin. Reversible modification of the terminal tails of core histones constitutes the major epigenetic mechanism for remodeling higher-order chromatin structure and controlling gene expression. HDAC inhibitors block this action and can result in hyperacetylation of histones, thereby affecting gene expression. It is this effect that allows HDAC inhibitors to reactivate dormant proviruses.

The “classical” HDAC inhibitors act exclusively on Class I and Class II HDACs by binding to the zinc-containing catalytic domain of the HDACs. These classical HDAC inhibitors fall into several groupings, in order of decreasing potency:

hydroxamic acids (or hydroxamates), such as trichostatin A, cyclic tetrapeptides (such as trapoxin B), and the depsipeptides,

benzamides,

electrophilic ketones, and

aliphatic acid compounds such as phenylbutyrate and valproic acid.

“Second-generation” HDAC inhibitors include the hydroxamic acids vorinostat (SAHA), belinostat (PXD101), LAQ824, and panobinostat (LBH589); and the benzamides: entinostat (MS-275), CI994, and mocetinostat (MGCD0103). The sirtuin Class III HDACs are dependent on NAD+ and are, therefore, inhibited by nicotinamide, as well derivatives of NAD, dihydrocoumarin, naphthopyranone, and 2-hydroxynaphaldehydes.

Vorinostat (rINN) or suberoylanilide hydroxamic acid (SAHA) is a member of a larger class of compounds that inhibit histone deacetylases (HDAC). Histone deacetylase inhibitors (HDAC inhibitors) have a broad spectrum of epigenetic activities. Vorinostat has been shown to bind to the active site of histone deacetylases and act as a chelator for Zinc ions also found in the active site of histone deacetylases Vorinostat's inhibition of histone deacetylases results in the accumulation of acetylated histones and acetylated proteins, including transcription factors crucial for the expression of genes needed to induce cell differentiation.

Panobinostat (LBH-589) (Novartis) is an experimental drug developed by Novartis for the treatment of various cancers. It is a hydroxamic acid and acts as a non-selective histone deacetylase inhibitor (HDAC inhibitor). Panobinostat inhibits multiple histone deacetylase enzymes, a mechanism leading to apoptosis of malignant cells via multiple pathways. Panobinostat is currently undergoing a phase I/II HIV treatment trial at a dosage of 20 mg/day on days 1, 3, 5 every other week for a period of 8 weeks (NCT01680094).

Romidepsin

In the study reported by Wei et al. in PLoS Pathog 10(4): e1004071. doi:10.1371/journal.ppat.1004071, the ability of romidepsin (RMD), a histone deacetylase inhibitor approved in the United States for the treatment of T-cell lymphomas, was tested for its ability to activate the expression of latent HIV. In an in vitro T-cell model of HIV latency, RMD was the most potent inducer of HIV (EC₅₀=4.5 nM) compared with vorinostat (VOR; EC₅₀=3,950 nM) and other histone deacetylase (HDAC) inhibitors in clinical development including panobinostat (PNB; EC₅₀=10 nM). The HIV induction potencies of RMD, VOR, and PNB paralleled their inhibitory activities against multiple human HDAC isoenzymes. In both resting and memory CD4 T cells isolated from HIV-infected patients on suppressive combination antiretroviral therapy (cART), a 4-hour exposure to 40 nM RMD induced a mean 6-fold increase in intracellular HIV RNA levels, whereas a 24-hour treatment with 1 μM VOR resulted in 2- to 3-fold increases. RMD-induced intracellular HIV RNA expression persisted for 48 hours and correlated with sustained inhibition of cell-associated HDAC activity. By comparison, the induction of HIV RNA by VOR and PNB was transient and diminished after 24 hours. RMD also increased levels of extracellular HIV RNA and virions from both memory and resting CD4 T-cell cultures. The activation of HIV expression was observed at RMD concentrations below the drug plasma levels achieved by doses used in patients treated for T-cell lymphomas.

Belinostat (PXD101) is a histone deacetylase inhibitor for the treatment of hematological malignancies and solid tumors. Belinostat is a HDAC inhibitor affecting class I and II HDACs. Belinostat is administered orally and IV. IV is infused at 400 mg/m² per day. Belinostat is administered orally at 500 mg/m² or 1000 mg/m² once or twice daily.

Aclarubicin (INN) or Aclacinomycin A is an anthracycline drug that is used in the treatment of cancer. Soil bacteria Streptomyces galilaeus can produce aclarubicin. The iv dosage initially is 175-300 mg/m², divided over 3-7 consecutive days, with a maintenance dose of 25-100 mg/m² 3-4 weekly.

Antibody b12 is a HIV-1 gp120 monoclonal antibody obtained as a Fab fragment by selection against MB gp120 from an antibody phage display library prepared from bone marrow of a long term asymptomatic HIV-1 seropositive donor. Antibody b12 is administered IV weekly at 1 mg/kg.

Aphidicolin is defined as a tetracyclic diterpene antibiotic with antiviral and antimitotical properties. Aphidicolin is a reversible inhibitor of eukaryotic nuclear DNA replication. It blocks the cell cycle at early S phase. It is a specific inhibitor of DNA polymerase A,D in eukaryotic cells and in some viruses and an apoptosis inducer in HeLa cells. Natural aphidicolin is a secondary metabolite of the fungus Nigrospora oryzae.

Apicidin is a HDAC inhibitor affecting class I HDACs. Apicidin is administered orally daily at 10 mg/kg.

BIX-01294, a diazepin-quinazolinamine derivative, is a histone-lysine methyltransferase (HMTase) inhibitor that modulates the epigenetic status of chromatin. BIX-01294 inhibits the G9aHMTase dependent levels of histone-3 lysine (9) methylation (H3K9me).

BML-210 is a histone deacetylase inhibitor. Treatment of A549 cells with BML-210 results in a dose-dependent increase in acetylated histone levels (EC50=36 μM). In HeLa extracts, the IC50 for inhibition of HDAC activity is 80 μM.

BMS-936558 is an antibody against PD-1, a protein involved in repressing the immune system. Blocking PD-1 with an antibody activates the immune system and enables it to fight tumors. BMS-936558 is administered IV at 3 mg/kg or 10 mg/kg at two or three week intervals.

Bryostatin-1 is a macrocyclic lactone isolated from the bryozoan Bugula neritina with antineoplastic activity. Bryostatin-1 binds to and inhibits the cell-signaling enzyme protein kinase C, resulting in the inhibition of tumor cell proliferation, the promotion of tumor cell differentiation, and the induction of tumor cell apoptosis. This agent may act synergistically with other chemotherapeutic agents. Bryoststin-1 is administered IV at 25 μg/m² or 40 μg/m² per day.

CG05/CG06 is a HDAC inhibitor. CG05/CG06 is administered at 0.15 μM or 0.3 μM.

Chaetocin is a fungal metabolite with antimicrobial and cytostatic activity. Chaetocin is a specific inhibitor of the lysine-specific histone methyltransferase SU(VAR)3-9 (IC₅₀=0.6 μM) of Drosophila melanogaster and of its human ortholog (IC₅₀=0.8 μM), and acts as a competitive inhibitor for S-adenosylmethionine.

CI-994 (Tacedinaline, PD-123654, GOE-5549, Acetyldinaline) is an orally active compound with a wide spectrum of antitumor activity in preclinical models, in vitro and in vivo. CI-994 is an inhibitor of Class I and II HDACs. CI-994 is administered orally daily at 500 mg/kg or 600 mg/kg.

Cytarabine is a nucleoside analog that interferes with nucleic acid replication. Cytarabine is administered IV or subcutaneously at 100 mg/m² per day.

Dactinomycin (actinomycin D, Cosmegen, Act-D) is the most significant member of actinomycines, which are a class of polypeptide antibiotics isolated from soil bacteria of the genus Streptomyces. Dactinomycin is administered IV daily at 15 μg/kg per day or 400 μg/m² per day.

Dihydrocoumarin is a compound found in Melilotus officinalis (sweet clover) that is commonly added to food and cosmetics. Dihydrocoumarin is an HDAC inhibitor that disrupts heterochromatic silencing.

Dihydrocoumarin is administered orally.

Disulfiram (Antabuse) is administered orally at 250 mg or 500 mg daily.

Droxinostat is a HDAC inhibitor affecting class III HDACs. Droxinostat selective inhibits HDAC3, 6, and 8, with IC50 values of 16.9 μM, 2.47 μM, and 1.46 μM, respectively, without inhibiting other HDAC members (IC50>20 μM). Droxinostat is administered IV or IM at 20 or 40 μM.

Entinostat (MS-275) is an inhibitor of HDAC (histone deacetylase) that preferentially inhibits HDAC1 (IC50=300 nM) over HDAC3 (IC50=8 μM). However, MS-275 does not inhibit HDAC8 (IC50>100 μM). Entinostat is administered orally at 10 mg or 15 mg once per day.

Givinostat (ITF2357) is a PAN HDAC inhibitor. Givinostat is administered orally once or twice daily at 50 mg or 100 mg (Rowinsky, et al. JCO December 1986 4 (121:1835-1844).

Hexamethylene bisacetamide (HMBA) at a dose from 4.8 to 33.6 g/m2/d

Oxamflatin is a HDAC inhibitor affecting class I HDACs. Romidepsin (Celgene) is a HDAC inhibitor that affects class I HDACs.

Scriptaid is a PAN HDAC inhibitor. Sodium butyrate is a HDAC inhibitor affecting class I and IIa HDACs.

Suberohydroxamic acid (SBHA) is a competitive HDAC inhibitor that affects HDAC classes I and III. SBHA has been shown to cause cell differentiation, cell cycle arrest, and apoptosis. SBHA inhibits HDAC1 with an IC50=0.25 μM and HDAC3 with an IC50=0.3 μM.

Trichostatin A (TsA) is a PAN HDAC inhibitor. Valproic acid (VPA) is a PAN HDAC inhibitor.

Stimulator of CD8 T Cell Response to HIV

Stimulation of an effective response by naive T cells requires three signals: TCR engagement, costimulation/IL-2, and a third signal that can be provided by IL-12. IL-2 contributes to both primary and secondary expansion in memory CD8+T-cell differentiation. IL-2 is responsible for optimal expansion and generation of effector functions following primary antigenic challenge. As the magnitude of T-cell expansion determines the numbers of memory CD8 T cells surviving after pathogen elimination, these events influence memory cell generation. Moreover, during the contraction phase of an immune response where most antigen-specific CD8 T cells disappear by apoptosis, IL-2 signals are able to rescue CD8 T cells from cell death and provide a durable increase in memory CD8+T-cell counts. At the memory stage, CD8+T-cell frequencies can be boosted by administration of exogenous IL-2. Significantly, only CD8 T cells that have received IL-2 signals during initial priming are able to mediate efficient secondary expansion following renewed antigenic challenge. Thus, IL-2 signals during different phases of an immune response are important in optimizing CD8+T-cell functions, thereby affecting both primary and secondary responses of these T cells.

IL-12 family members are an important link between innate and adaptive immunity. IL-12 drives Th1 responses by augmenting IFN-gamma production, which is generally important for clearance of intracellular pathogens. IL-12 is the major cytokine influencing the level of IFN-gamma production by CD8 T cells. IL-12 promotes longer duration conjugation events between CD8 T cells and DC. IL-12 augments naive CD8 T cell activation by facilitating chemokine production, thus promoting more stable cognate interactions during priming. In addition to being required for acquisition of cytolytic function, IL-12 is required for optimal IL-2-dependent proliferation and clonal expansion. IL-12 stimulates expression of the IL-2R-chain (CD25) to much higher levels than are reached in response to just TCR and costimulation and/or IL-2. In addition, high CD25 expression is substantially prolonged in the presence of IL-12. As a consequence, the cells proliferate more effectively in response to low levels of IL-2. IL-2 and IL-12 both act to increase expression of both CD25 and the IL-12R, thus providing positive cross-regulation of receptor expression.

IL-15 in HIV-infected individuals can enhance the function, survival, and expansion of HIV-specific CD8 T cells. IL-15 is crucial for the development of naive and memory CD8 T cells and is delivered through a mechanism called transpresentation. For example, memory CD8 T cells grow more dependent on IL-15 transpresentation by dendritic cells. (Sneller et al., Blood., 2011 Dec. 22; 118(26):6845-8. Epub 2011 Nov. 8). IL-15 promotes activation and maintenance of natural killer (NK) and CD8 T effector memory (T(EM)) cells, making it a potential immunotherapeutic agent for the treatment of cancer and immunodeficiency states. IL-15 at a dose of 20 μg/kg/d administered by continuous intravenous infusion for 10 days resulted in a massive (100-fold) expansion of CD8 T(EM) cells in the peripheral blood. In contrast, the administration of 20-40 μg/kg/d of IL-15 by subcutaneous injection resulted in a more modest (10-fold) expansion of CD8 T(EM) cells. NK expansion was similar in both the continuous intravenous and daily subcutaneous treatment groups. IL-15 administered by continuous intravenous infusion is able to induce markedly greater expansions of CD8 T(EM) cells than the same dose administered by other routes.

Formulation of Compositions

The compounds and compositions disclosed herein can be formulated in any useful way. Generally, the nature of the compound and the route of administration will influence the choice of formulation.

In one embodiment, the inhibitors of HIV infection of CD4 T cells and stimulator of reactivation of latent HIV can be administered together in a single composition. In one embodiment, the inhibitors and reactivation stimulator are administered in separate compositions. In one embodiment, the first and second inhibitors of HIV infection of CD4 T cells are administered together in a single composition while the reactivation stimulator is administered in a separate composition. In one embodiment, the first inhibitor of HIV infection of CD4 T cells and the reactivation stimulator are administered together in a single composition while the second inhibitor of HIV infection of CD4 T cells is administered in a separate composition. In one embodiment, the second inhibitor of HIV infection of CD4 T cells and the reactivation stimulator are administered together in a single composition while the first inhibitor of HIV infection of CD4 T cells is administered in a separate composition.

The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject in need of treatment of HIV infection or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

Any of the compounds disclosed herein can be used therapeutically in combination with a pharmaceutically acceptable carrier. The compounds described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compounds described herein. These most typically would be standard carriers for administration of compositions to humans. Other compounds can be administered according to standard procedures used by those skilled in the art.

The pharmaceutical compositions described herein can include, but are not limited to, carriers, thickeners, diluents, buffers, preservatives, surface active agents and the like in addition to the molecule of choice. Generally, oral administration is preferred and is generally available for the compounds and compositions disclosed herein. Parenteral administration, if used, is generally characterized by injection. Injectables can be prepared in conventional forms, either as liquid solutions or suspensions, solid forms suitable for solution or suspension in liquid prior to injection, or as emulsions. Parenteral administration can use a slow release or sustained release system such that a constant dosage is maintained.

### III. METHODS OF TREATMENT

The disclosed compounds and compositions can be administered in any manner or route suitable to the compound or composition and the formulation of the compound or composition. Such techniques are well-known and can be applied to the methods and compositions disclosed herein.

Courses of Treatment

The methods and compositions can be used in courses of treatment in order to achieve clinical or other goals. Generally, the compositions can be administered over periods of time measured in weeks and months. Viral infections such as HIV are generally affected by treatments over similar time periods. Reactivation of latent virus and subsequent clearing of infected cells generally requires weeks to months of treatment. In particular, reactivation and clearance of the small number of infected cells remaining after the beginning and middle of treatment requires time. Reactivation of latent virus and clearance of infected cells can be conceptualized as occurring via half-life kinetics based on a rate constant. A course of treatment generally should last long enough to reduce remaining latently and/or actively infected cells to below a threshold level. Such clinical factors and their assessment are well known and are discussed elsewhere herein.

The schedule of treatment during a course of treatment generally can be a schedule of treatment that will keep the compounds or compositions at or above an effective, therapeutic, or useful level in the subject. However, reactivation of latent virus and clearance of infected cells generally does not require that constant levels of the compounds or compositions. Rather, the levels need only be sufficient to reduce the half-life of latent virus and/or infected cells and to reduce the possibility of new cell infection and of establishment of a provirus in a cell.

As with most therapies, a consistent schedule and fewer administrations are preferred to irregular schedules and frequent administrations. However, as is well-known, the half-life of therapeutic compounds and compositions in subjects generally determine the frequency of administration. For the disclosed methods and compositions, the schedule of administration generally will be one or more administrations per day of the compositions.

In one embodiment, the disclosed compositions can be administered from 10 to 80 weeks, preferably from 10 to 40 weeks, more preferably from 10 to 30 weeks, and most preferably from 20 to 40 weeks. In a particular embodiment, the period of time can end after the earlier of 40 weeks or 4 weeks after HIV infected cells are no longer detected, preferably 3 weeks after HIV infected cells are no longer detected, most preferably 2 weeks after HIV infected cells are no longer detected. In another particular embodiment, the period of time can end after the earlier of 40 weeks or 4 weeks after the HIV viral load becomes undetectable, preferably 3 weeks after the HIV viral load becomes undetectable, most preferably 2 weeks after the HIV viral load becomes undetectable.

These compounds can be administered alone or in various combinations. In one embodiment, the inhibitors of HIV infection of CD4 T cells can be administered one to four times daily, preferably one to three times daily, more preferably one or two times daily, most preferably one time daily. In one embodiment, the inhibitors of HIV infection of CD4 T cells can be administered one to four times daily, preferably one to three times daily, more preferably one or two times daily, most preferably one time daily. In one embodiment, the stimulator of reactivation of latent HIV can be administered one to four times daily, preferably one to three times daily, more preferably one or two times daily, most preferably one time daily. In one embodiment, the highly active antiretroviral therapy (HAART) can be administered one to four times daily, preferably one to three times daily, more preferably one or two times daily, most preferably one time daily. In one embodiment, the stimulator of CD8 T cell response to HIV can be administered one to four times daily, preferably one to three times daily, more preferably one or two times daily, most preferably one time daily.

Different compounds and compositions can be administered following the same schedule, a similar schedule, or different schedules. For example, courses of treatment of different compounds and compositions can be overlapping, completely overlapping, partially overlapping, or sequential. In one embodiment, the highly active antiretroviral therapy (HAART), stimulator of CD8 T cell response to HIV, or both, can be administered simultaneous with, overlapping with, or following the administration of the inhibitors of HIV infection of CD4 T cells and the stimulator of reactivation of latent HIV.

The methods and compositions can be used with any virally infected subject. In one embodiment, the subject is receiving anti-HIV therapy. In another embodiment, the subject is naïve of anti-HIV therapy or on an anti-HIV therapy holiday. In a particular embodiment, the subject has not been administered any anti-HIV treatment for at least 2 weeks prior to beginning a course of treatment of the methods or compositions disclosed herein, preferably for at least 3 weeks, more preferably for at least 4 weeks, most preferably for at least 5 weeks, and in one embodiment, for at least 10 weeks prior to administration of the inhibitors and reactivation stimulator. In one embodiment, the subject is not administered HAART for at least the first 10 weeks of the start of a course of treatment disclosed herein, preferably for at least the first 15 weeks, more preferably for at least the first 20 weeks, most preferably for at least the first 30 weeks.

In one embodiment, the inhibitors of HIV infection of CD4 T cells and stimulator of reactivation of latent HIV are administered in the same course of treatment. In one embodiment, the inhibitors and reactivation stimulator are administered in different courses of treatment. In one embodiment, the first and second inhibitors of HIV infection of CD4 T cells are administered in the same course of treatment while the reactivation stimulator is administered in a different course of treatment. In one embodiment, the first inhibitor of HIV infection of CD4 T cells and the reactivation stimulator are administered in the same course of treatment while the second inhibitor of HIV infection of CD4 T cells is administered in a different course of treatment. In one embodiment, the second inhibitor of HIV infection of CD4 T cells and the reactivation stimulator are administered in the same course of treatment while the first inhibitor of HIV infection of CD4 T cells is administered in a different course of treatment. In one embodiment, the inhibitors and reactivation stimulator can be administered in different course of treatment from the highly active antiretroviral therapy (HAART), the stimulator of CD8 T cell response to HIV, or both.

Assessing Effectiveness of Treatment

The effectiveness of the methods and compositions can be assessed in any suitable manner. The effect of the methods and compositions on subjects in which they are used is a preferred approach. For example, the methods and courses of treatment can be assessed by testing one or more clinical factors. For assessment of treatments of HIV infections, such assessments can include, for example, CD4 T cell count, HIV viral load, and HIV infected cell count. Any other assessment of the state of HIV infection can also be used.

The methods and courses of treatment can also be assessed and adjusted based on assessments of the state of viral infection. For example, methods and courses of treatment in the methods can be continued for one or more clinical endpoints and/or until one or more clinical factors have reached a threshold level. For example, a course of treatment can be continued until CD4 T cell count has increased to or above a threshold level, HIV viral load has decreased to or below a threshold level, and/or HIV infected cell count has decreased to or below a threshold level. In particular embodiments, a course of treatment can be continued until: CD4 T cell count has increased to or above 300 per cubic millimeter, preferably 500 per cubic millimeter; until HIV viral load has decreased to or below 1000 copies per milliliter of blood, preferably 100 copies per milliliter of blood, most preferably undetectable; and/or until HIV infected cell count has decreased to or below 1% of peripheral blood mononuclear cells, preferably below 0.1% of peripheral blood mononuclear cells, most preferably below 0.01% of peripheral blood mononuclear cells.

The methods and compositions can result in an improved state of viral infection. For example, the methods and compositions can result in an improved state of viral infection for a period of time following the end of a course of treatment. For example, CD4 T cell count can remain at or above a threshold level, HIV viral load can remain at or below a threshold level, and/or HIV infected cell count can remain at or below a threshold level for and/or at 8 weeks, preferably 3 months, more preferably 6 months, and most preferably 12 months following the end of a course of treatment. In particular embodiments, CD4 T cell count can remain at or above 300 per cubic millimeter, preferably 500 per cubic millimeter; HIV viral load can remain at or below 1000 copies per milliliter of blood, preferably 100 copies per milliliter of blood, most preferably undetectable; and/or HIV infected cell count can remain at or below 1% of peripheral blood mononuclear cells, preferably below 0.1% of peripheral blood mononuclear cells, most preferably below 0.01% of peripheral blood mononuclear cells for and/or at 8 weeks, preferably 3 months, more preferably 6 months, and most preferably 12 months following the end of a course of treatment.

Clinical factors of HIV infection generally can be assessed in blood or blood components. However, in some embodiments, clinical factors can be assessed in other types of samples, such as semen, vaginal secretions, gut-associated lymphoid tissue (GALT), bone marrow, saliva, lymphatic fluid, lymph tissue, and cerebrospinal fluid.

In many embodiments, it is expected that clinical factors will improve further beyond the end of the method or course of treatment. This is expected because, for example, the clinical factors can lag the primary effects of the methods and courses of treatment.

As used herein, “effective” means that the viral load of the patient remains suppressed following discontinuation of treatment for at least two weeks, one month, two months, or longer. This can be determined using any of the foregoing methods, but typically is performed by measuring the amount of virus in the blood.

Subject Selection and Pretreatment

Any subject in need of the disclosed methods and compositions can be treated. Generally, suitable subjects are infected with HIV or have been exposed to HIV. Subjects can be, for example, newly infected, infected long-term, anti-HIV therapy experienced, or naïve to anti-HIV therapy. In some embodiments, the method can be performed on subjects that have not been administered any anti-HIV treatment. This state may make the subject more receptive to the method and make one or more of the compounds used more effective. Subjects generally should be selected for such appropriate characteristics. Such selection and considerations are well known regarding HIV therapies.

The present invention will be further understood by reference to the following non-limiting examples. Examples 2-5 demonstrate combination therapies that should be effective in maintaining low viral load after cessation of drug therapy as defined above, as well as combinations that are not effective.

### Example 1

### Simulation of HIV infection Treatment Outcome and Correlation with Multiple Clinical Trials

A computer model of the human immune system has been developed which can accurately simulate the effect on the immune system and clinical factors of HIV infection and clinical treatments of HIV infection.

The model has been validated by inputting the drugs, dosages, and dosing regimens as well as patients to be treated, for drugs in which the clinical outcomes have been described in the literature. The results obtained with the computer model, which is not based on input of the clinical trial results to be validated, demonstrate that the treatments using reverse transcriptase inhibitors do not result in elimination of HIV reservoirs, as shown by a rapid rise in blood viral load following cessation of drug treatment.

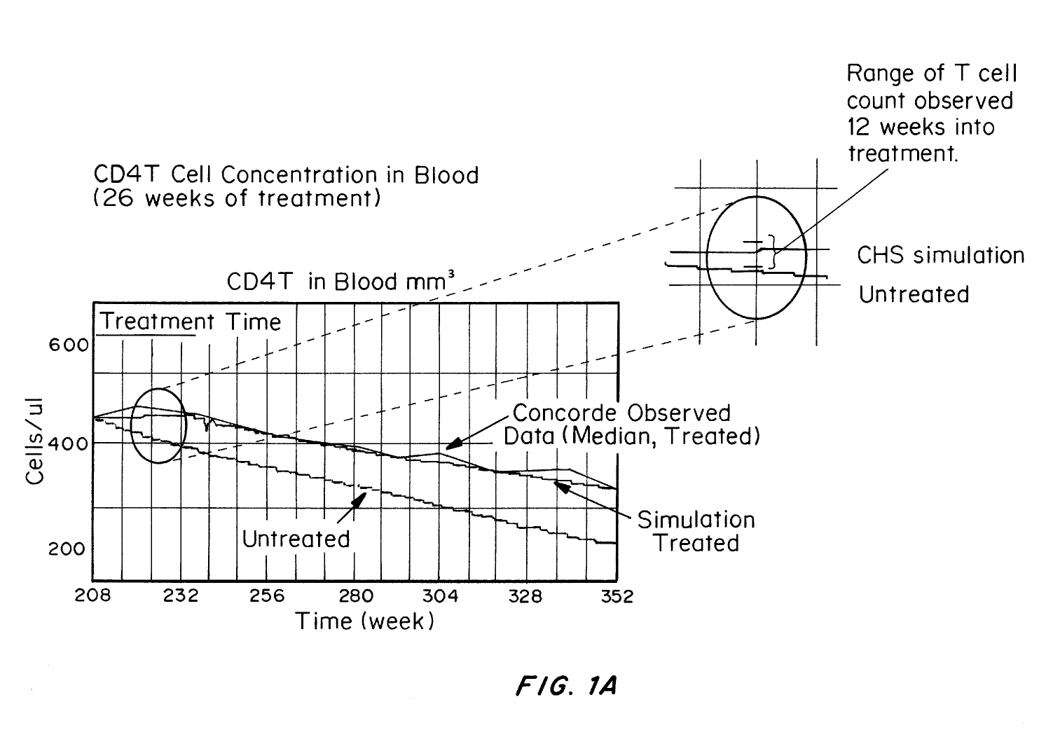

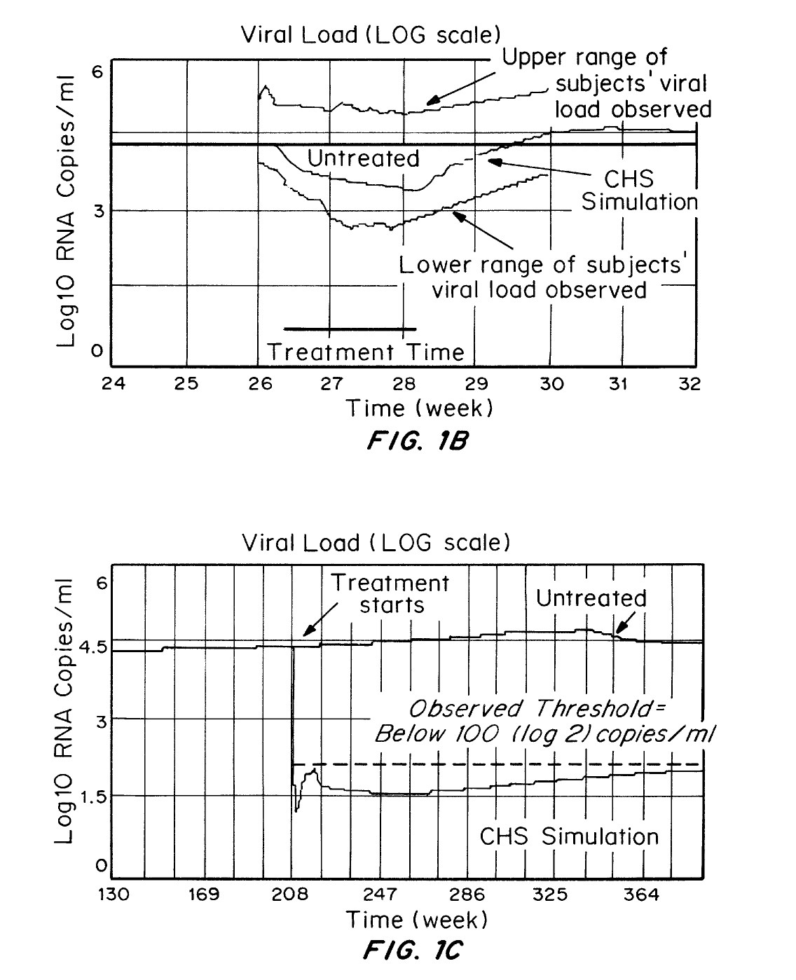

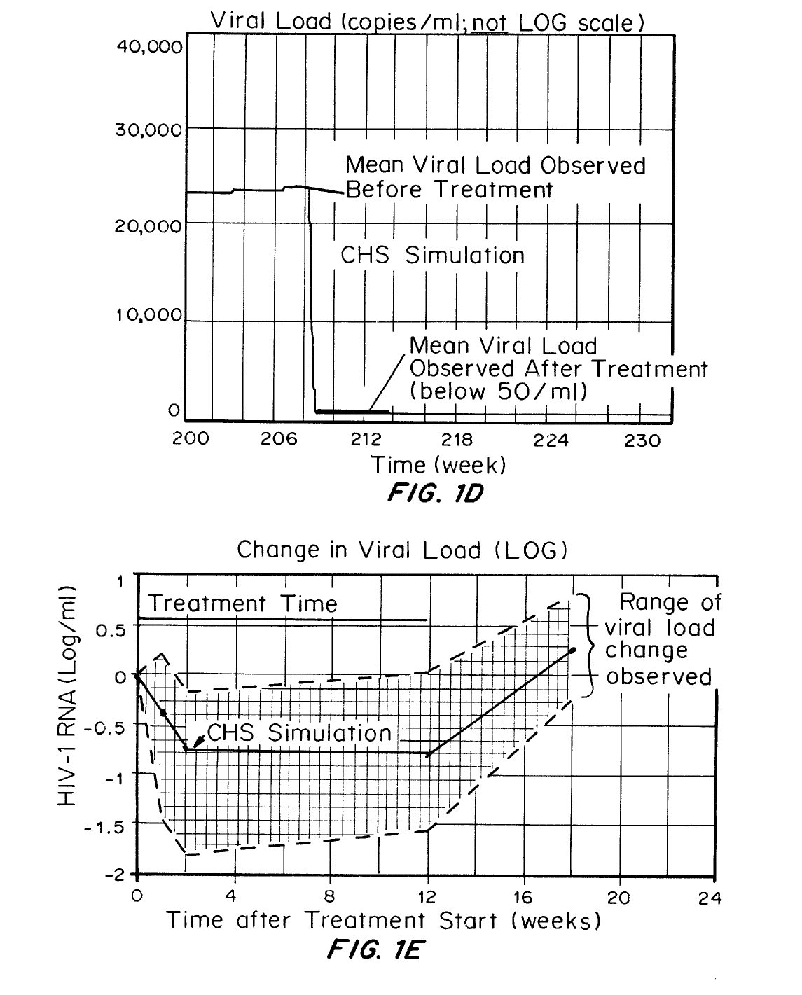

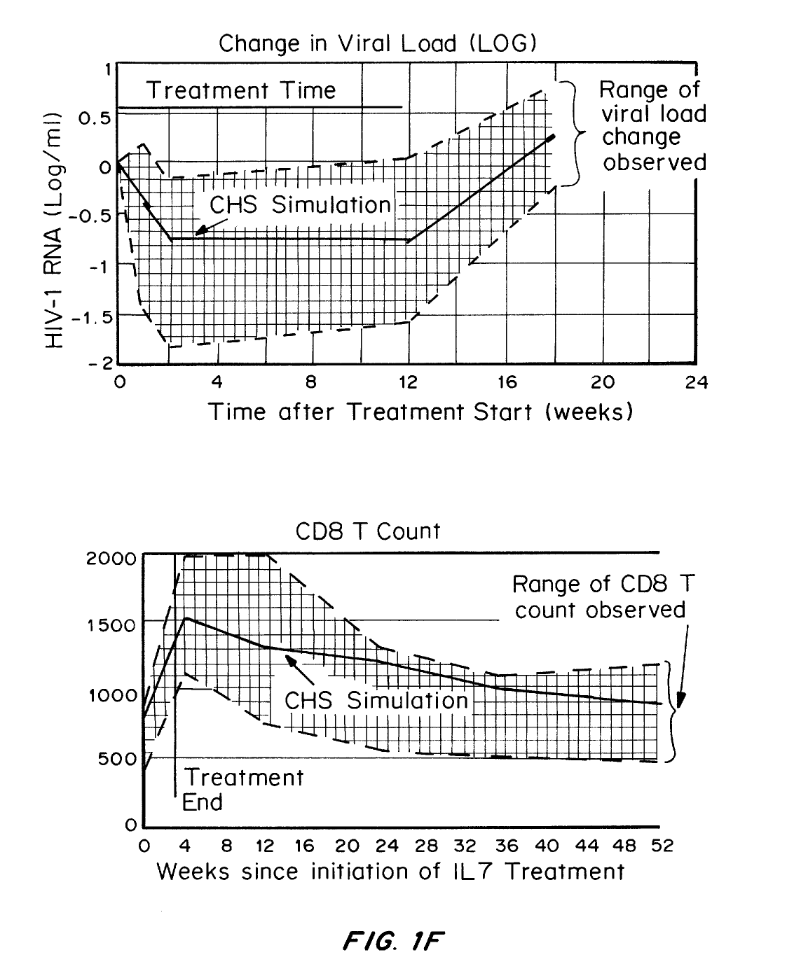

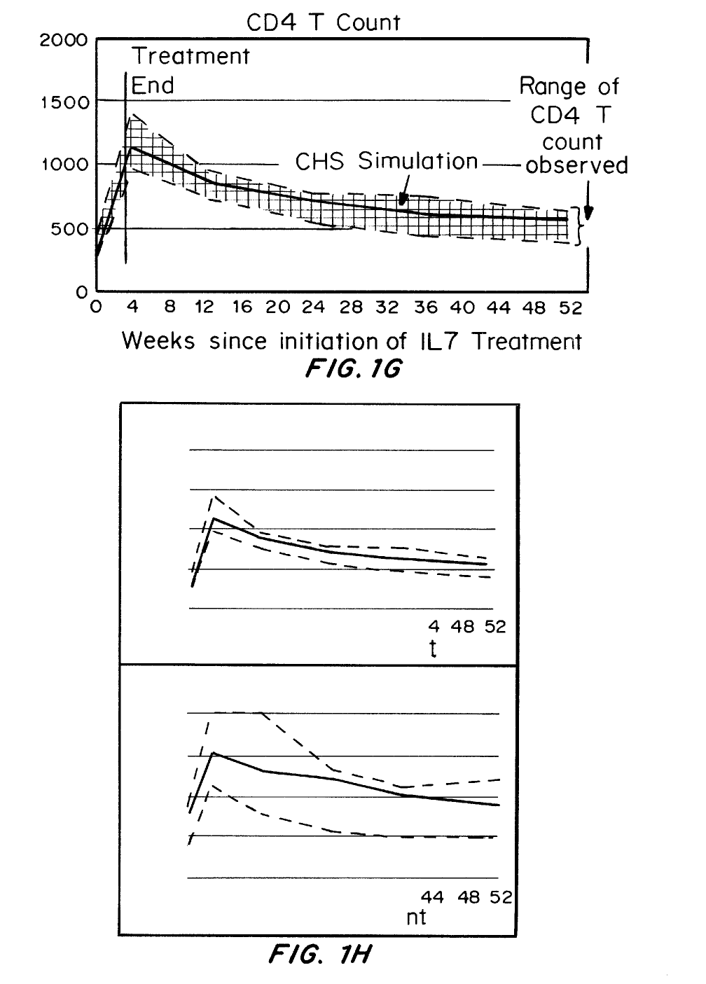

Seven active HIV drug trials were modeled based on patients being treated, drugs, dosages, and treatment regimens. Results of actual outcomes compared to simulated results are shown in FIGS. 1A-1H.

A. AZT: Concorde Trial

This study was reported in Lancet., 1994 Apr. 9; 343(8902):871-81.

Concorde was a double-blind randomised comparison of two policies of zidovudine treatment in symptom-free individuals infected with human immunodeficiency virus (HIV): (a) immediate zidovudine from the time of randomisation (Imm); and (b) deferred zidovudine (Def) until the onset of AIDS-related complex (ARC) or AIDS (CDC group IV disease) or the development of persistently low CD4 cell counts if the clinician judged that treatment was indicated. Between October, 1988, and October, 1991, 1749 HIV-infected individuals from centers in the UK, Ireland, and France were randomly allocated to zidovudine 250 mg four times daily (877 Imm) or matching placebo (872 Def). Follow-up was to death or Dec. 31, 1992 (total 5419 person-years; median 3.3 years) and only 7% of the 1749 had not had a full clinical assessment after Jul. 1, 1992. Of those allocated to the Def group, 418 started zidovudine at some time during the trial, 174 (42%) of them at or after they were judged by the clinician to have developed ARC or AIDS (nearly all confirmed subsequently) and most of the remainder on the basis of low CD4 cell counts. There was no statistically significant difference in clinical outcome between the two therapeutic policies. The 3-year estimated survival probabilities were 92% (95% CI 90-94%) in Imm and 94% (92-95%) in Def (log-rank p=0.13), with no significant differences overall or in subgroup analyses by CD4 cell count at baseline. Similarly, there was no significant difference in progression of HIV disease: 3-year progression rates to AIDS or death were 18% in both groups, and to ARC, AIDS, or death were 29% (Imm) and 32% (Def) (p=0.18), although there was an indication of an early but transient clinical benefit in favour of Imm in progression to ARC, AIDS, or death. However, there was a clear difference in changes in CD4 cell count over time in the two groups.

Results comparing actual versus predicted results are shown in FIG. 1A. AZT, a “classic” HIV drug, inhibits HIV replication in target cells by inhibiting reverse transcription of the virus. The treatment used 250 mg AZT, 4 times daily, for 6 months. CD4T cell count was monitored.

The simulation is an extremely accurate predictor of the median impact observed in Concorde trial—both the quantum and the timing, falling within the range of impact observed at 3 months into treatment using 300 mg AZT 2 times daily for 13 days (trial stopped).

B. AZT: Ruane Trial

In 1985, 3′-azido-thymidine (AZT, zidovudine) was identified as the first nucleoside analog with activity against human immunodeficiency virus type 1 (HIV-1) (Mitsuya et al., 1985, 1987; Mitsuya & Broder, 1986), the etiologic agent of acquired immunodeficiency syndrome (Barre-Sinoussi et al., 1983; Gallo et al., 1984). The initial phase 1 clinical trial of AZT at the NCI, in collaboration with the scientists from Burroughs-Wellcome and Duke University proved that the drug could be safely administered to patients with HIV, that it increased their CD4 counts, restored T cell immunity as measured by skin testing, and that it showed strong evidence of clinical effectiveness, such as inducing weight gain in AIDS patients. It also showed that levels of AZT that worked in the test tube could be injected into patients in serum and suppository form, and that the drug penetrated deeply only into infected brains. This study showed that HIV-1 replication could be suppressed by small molecule chemotherapeutic agents. Zidovudine was approved by the United States of America Food and Drug Administration for the treatment of HIV-1 infection in 1987.

As demonstrated by FIG. 1B, the Ruane trial monitored (a) viral load in bloodstream, as reduced by treatment, and (b) the viral load rebound after treatment ended.

The simulation exhibits the same time pattern and the same magnitude of impact on the viral load as was observed during and after the treatment. The simulation shows return to untreated viral set point within 2 weeks of ending treatment, just as was observed in the trial results.

C. 2 NRTI+NNRTI: Gallant Trial

Gallant et al. (N Engl. J. Med. 2006 Jan. 19; 354(3):251-60) reported on an open-label, noninferiority study involving 517 patients with HIV infection who had not previously received anti-retroviral therapy and who were randomly assigned to receive either a regimen of tenofovir disoproxil fumarate (DF), emtricitabine, and efavirenz once daily (tenofovir-emtricitabine group) or a regimen of fixed-dose zidovudine and lamivudine twice daily plus efavirenz once daily (zidovudine-lamivudine group). The primary end point was the proportion of patients without baseline resistance to efavirenz in whom the HIV RNA level was less than 400 copies per milliliter at week 48 of the study. Through week 48, significantly more patients in the tenofovir-emtricitabine group reached and maintained the primary end point of less than 400 copies of HIV RNA per milliliter than did those in the zidovudine-lamivudine group (84 percent vs. 73 percent, respectively; 95 percent confidence interval for the difference, 4 to 19 percent; P=0.002).

HAART combines two nucleoside/nucleotide reverse-transcription inhibitors (NRTIs) and one non-nucleoside reverse-transcription inhibitor (NNRTI), thus reducing viral integration in the target cell. Viral load in bloodstream was monitored, with treatment reducing the load to less than 2 log (<100) copies/ml, just as the model simulated, as shown in FIG. 1C.

D. 2 NRTI+Protease Inhibitor: Gemini Trial

Wamsley, et al., reported in J. Acquir. Immune Defic. Syndr., 2009 Apr. 1; 50(4):367-74 on the results of a 48-week, randomized, open-label, 2-arm study was conducted by Hoffman-La Roche to compare the efficacy of saquinavir/ritonavir BID plus emtricitabine/tenofovir QD versus lopinavir/ritonavir BID plus emtricitabine/tenofovir QD in treatment-naïve HIV-1 infected patients and to evaluate the efficacy, safety and tolerability of saquinavir/ritonavir or lopinavir/ritonavir in combination with emtricitabine/tenofovir in patients with HIV-1 infection who have received no prior HIV treatment. Patients were randomized to receive either saquinavir/ritonavir 1000/100 mg po bid+emtricitabine/tenofovir 200/300 mg po qd, or lopinavir/ritonavir 400/100 mg po bid+emtricitabine/tenofovir 200/300 mg po qd.

A similar proportion of participants in the SQV/r (n=167) and LPV/r (n=170) arms had HIV-1 RNA levels <50 copies per milliliter at week 48: 64.7% vs 63.5% and estimated difference in proportion for noninferiority: 1.14%, 96% confidence interval: −9.6 to 11.9 (P<0.012), confirming that SQV/r was noninferior to LPV/r treatment. There were no significant differences in week 48 CD4 counts between arms. The rate and severity of adverse events were similar in both groups. There were no significant differences in the median change from baseline between arms in plasma lipids except for triglyceride levels, which were significantly higher in the LPV/r at week 48.

In treatment-naive, HIV-1-infected patients, SQV/r treatment was noninferior in virologic suppression at 48 weeks to LPV/r treatment and offered a better triglyceride profile.

2 NRTIs and a protease inhibitor (reducing viral replication) constitute another current standard treatment. The impact of treatment was observed in the trial to reduce the mean viral load in bloodstream to less than 50 copies/ml, again, as predicted by the CHS simulation, shown in FIG. 1D.

E. Interferon Alpha: Asmuth Trial

Asmuth, et al., reported in J. Infect. Dis., 2010 Jun. 1; 201(11):1686-96 a study of the antiviral activity of pegylated interferon alfa-2a in participants with untreated human immunodeficiency virus type 1 (HIV-1) infection without chronic hepatitis C virus (HCV) infection. Untreated HIV-1-infected volunteers without HCV infection received 180 microg of pegylated interferon alfa-2a weekly for 12 weeks. Changes in plasma HIV-1 RNA load, CD4(+) T cell counts, pharmacokinetics, pharmacodynamic measurements of 2′,5′-oligoadenylate synthetase (OAS) activity, and induction levels of interferon-inducible genes (IFIGs) were measured. Nonparametric statistical analysis was performed.

Eleven participants completed 12 weeks of therapy. The median plasma viral load decrease and change in CD4(+) T cell counts at week 12 were 0.61 log(10) copies/mL (90% confidence interval [CI], 0.20-1.18 log(10) copies/mL) and −44 cells/microL (90% CI, −95 to 85 cells/microL), respectively. There was no correlation between plasma viral load decreases and concurrent pegylated interferon plasma concentrations. However, participants with larger increases in OAS level exhibited greater decreases in plasma viral load at weeks 1 and 2 (r=−0.75 [90% CI, −0.93 to −0.28] and r=−0.61 [90% CI, −0.87 to −0.09], respectively; estimated Spearman rank correlation). Participants with higher baseline IFIG levels had smaller week 12 decreases in plasma viral load (0.66 log(10) copies/mL [90% CI, 0.06-0.91 log(10) copies/mL]), whereas those with larger IFIG induction levels exhibited larger decreases in plasma viral load (−0.74 log(10) copies/mL [90% CI, −0.93 to −0.21 log(10) copies/mL]).

The results demonstrated that pegylated interferon alfa-2a was well tolerated and exhibited statistically significant anti-HIV-1 activity in HIV-1-monoinfected patients. The anti-HIV-1 effect correlated with OAS protein levels (weeks 1 and 2) and IFIG induction levels (week 12) but not with pegylated interferon concentrations.

The Asmuth trial tested Interferon alpha as a treatment for Hepatitis C. Interferon alpha hinders reverse transcription and replication of the virus. FIG. 1E compares the actual results with the impact simulated by the model. The same shape and timing was observed, with the simulation falling right in the middle of the range of results observed in the trial.

F. Interleukin 7: Levy (7) Trial

Levy, et al., Clin. Infect. Dis., 2012 July; 55(2):291-300. Epub 2012 May 1 showed that Interleukin 7 stimulates proliferation of naïve and central memory CD4 T and CD8 T cells. The Levy trial tested weekly injections of 10, 20 or 30 μg/kg of IL7, for 3 weeks, on HIV-positive individuals also on standard anti-retroviral treatment. The Levy trial measured CD8 T count (cells/μl) at 4, 12, 24, 36, and 52 weeks after initiation of the IL7 treatment. The increase in CD4 T and CD8 T counts were monitored.

As shown by FIG. 1E, the CHS simulation shows the same time pattern and magnitude of response, falling near the middle of the range of results observed in the trial, for both CD4 T and CD8 T cell counts.

G. Interleukin 2: Levy (2) Trial

This trial was reported by Levy, et al; ILIADE Study Group. Effect of intermittent interleukin-2 therapy on CD4+T-cell counts following antiretroviral cessation in patients with HIV. AIDS. (2012) 26(6):711-20. (NCT00071890).

The Levy (2) trial showed that Interleukin 2 stimulates proliferation of activated T cells. Levy tested three cycles of twice daily injections of 6 million IUs of interleukin-2 (cycles lasted five days each at weeks 0, 8 and 16) on HIV positive individuals also on standard anti-retroviral treatment (ART). Treatment was discontinued at week 24. Levy measured CD4 T cell counts every 8 weeks, during IL-2 therapy and subsequent cessation of ART for a total of 72 weeks.

The simulation results in FIG. 1G are nearly identical to the magnitude and timing of the observed change in median cell counts.

HIV TREATMENT MATRIX

| THERA- | | | | |
|---|---|---|---|---|
| PEUTIC | DRUG 1 | DRUG 2 | DRUG 3 | |
| REGIMEN | DOSAGE | DOSAGE | DOSAGE | DURATION |
| EXAMPLE 2¹ | Maraviroc | HDACi - | Cytokine IL-15 | Initiate at 26 |
| | | vorinostat | | weeks; |
| | | | | continue to |
| | | | | 40 weeks |
| EXAMPLE 3² | Maraviroc | HDACi - | | Initiate at 26 |
| | | vorinostat | | weeks; |
| | | | | continue to |
| | | | | 80 weeks |
| EXAMPLE 3³ | Maraviroc | HDACi - | HAART (two | Drugs 1 and 2 |
| | | vorinostat | non-nucleoside | for weeks 26- |
| | | | reverse | 36; then add |
| | | | transcriptase | drug 3 weeks |
| | | | inhibitors and | 34-46 |
| | | | one protease | |
| | | | inhibitor) | |
| EXAMPLE 5⁴ | Maraviroc | Hydroxyl | HDACi - | Week 26 to |
| | | chloro- | vorinostat | week 41 |
| | | quine | | |
| | | sulfate | | |

¹Treatment effective
²Treatment ineffective
³Treatment effective
⁴Treatment effective

### Example 2

### Simulated Treatment to Hinder CD4 T Cell Infection; Force Latently Infected Cells to Produce and Present HIV; and Push a Stronger CD8 T Cell Response to HIV

Method of Treatment

Treatment simulation was performed to target three points at the same time to hinder CD4 T cell infection; force latently infected cells to produce and present HIV; and push a stronger CD8 T cell response to HIV. In this simulation, new infections are held in check directly (as with a CCR5 inhibitor), latent cells are pushed out via activation (such as with a histone deacetylase inhibitors), and the CD8 T cell response is magnified (as IL-15 might accomplish), for example, by administering Maraviroc, vorinostat and IL-15 using standard dosing: Maraviroc at 600 mg/2×/daily, Vornisotat at 400 mg daily.

Under the specific treatment protocol tested, new infections are slowed by hindering CD4 T cell activation, therefore reducing the target population for HIV infection. Latently infected cells are forced out of latency, and the CD8 T cell response is increased with IL-15. This treatment protocol increases the attack against HIV while forcing all infected cells out in the open and at the same time holding new infections down.

Results

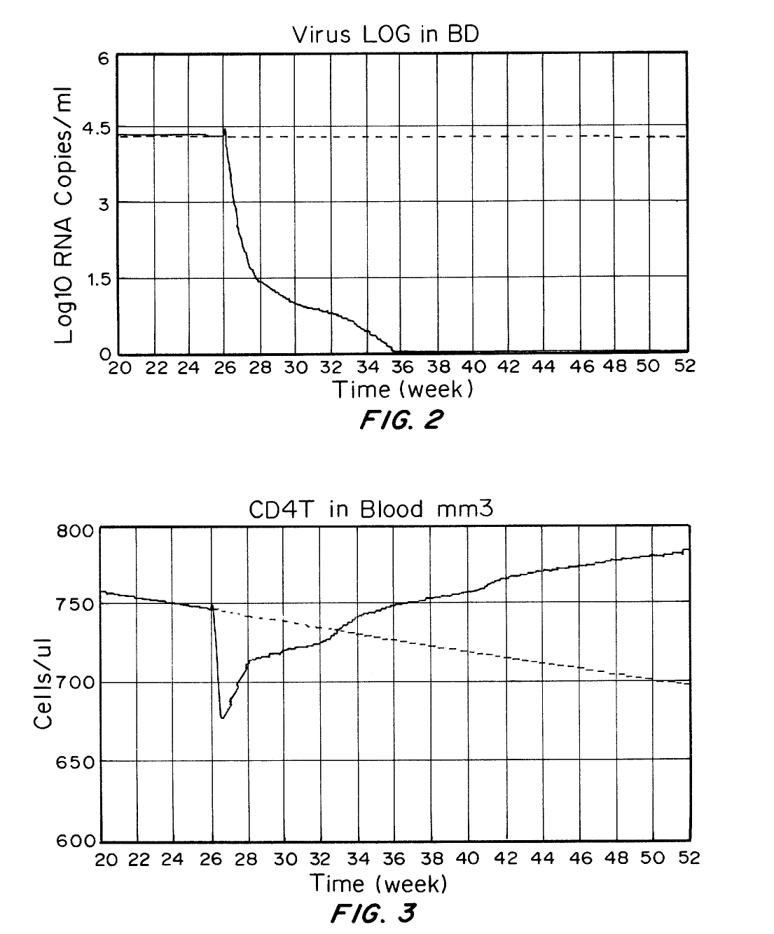

This strategy takes approximately one month to clear HIV in the simulation. FIGS. 2 and 3 show the results of a simulated treatment protocol being initiated at week 26 and continuing to week 40. FIG. 2 tracks the HIV viral load and shows that the HIV viral load in the blood approaches zero around week 36. FIG. 3 tracks CD4 T cell count and shows that CD4 T cell count increases during the course of treatment (for the duration of the simulation shown).

These treatment protocols show that HIV viral load can be pushed to undetectable levels and indicate that longer term success in affecting latent HIV infection can be achieved with more robust reactivation of latent HIV.

### Example 3

### Simulation of Combination Treatment to Reduce HIV Infection of CD4+T Cells, Drives HIV and Associated Antigen Presentation from Latently Infected Cells, and Prevents Viral Replication

Method of Treatment

This example describes simulations using the model of a treatment strategy that uses two targets (“levers”) concurrently, then adds a third, HAART, to clear the rest of the HIV. The initial targets are to reduce HIV infection of CD4+T cells and to drive HIV and associated antigen presentation from latently infected cells. The first effect can be accomplished with, for example, a CCR5 inhibitor such as Maraviroc. The second effect can be accomplished with, for example, a histone deacetylase inhibitors such as Vorinostat. All simulations using Vorinostat assume 400 mg, once daily and Maraviroc at 600 mg/2×/daily.

Results

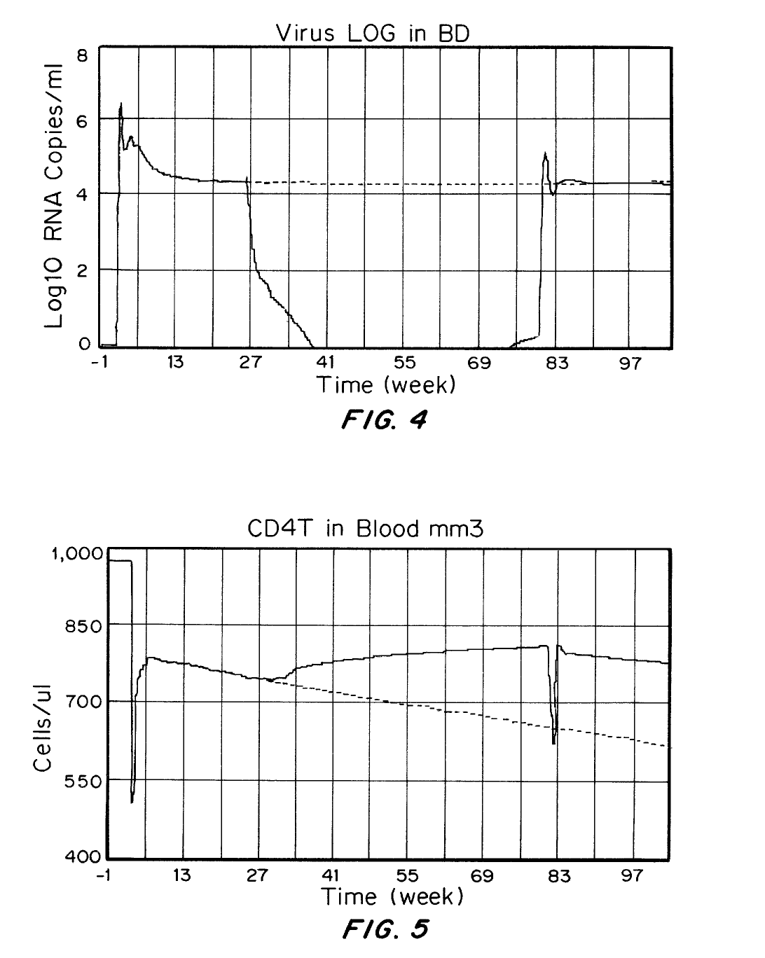

In the initial simulation runs with just the first two treatments, HIV is not cleared. The results shown in FIGS. 4 and 5 are for the treatment protocol initiated at the start of week 26 and ended treatment at the start of week 80. Over the first ten weeks, viral copies per ml drop effectively to zero and appear to be cleared (see FIG. 4). In this scenario, latently infected cells are completely eliminated in the first four weeks. However, a small population of infected cells is maintained in the GALT tissue causing viral load to reappear around week 75 and return to set-point when treatment is terminated. CD4 T cell counts increase during the treatment period but then began to decline after treatment termination (FIG. 5).

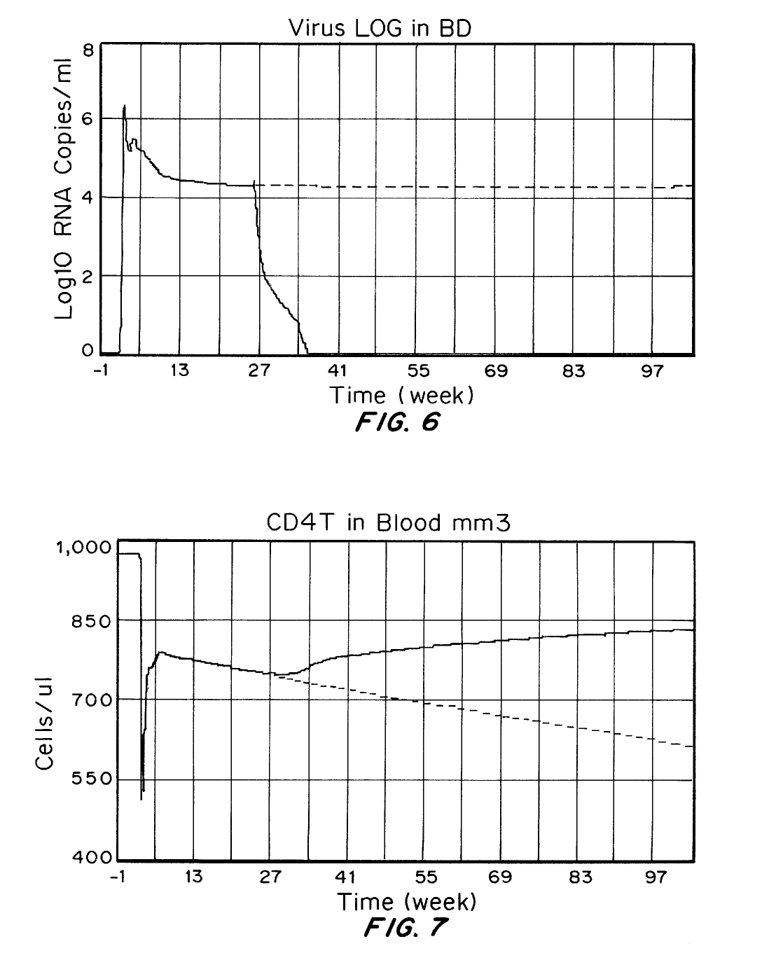

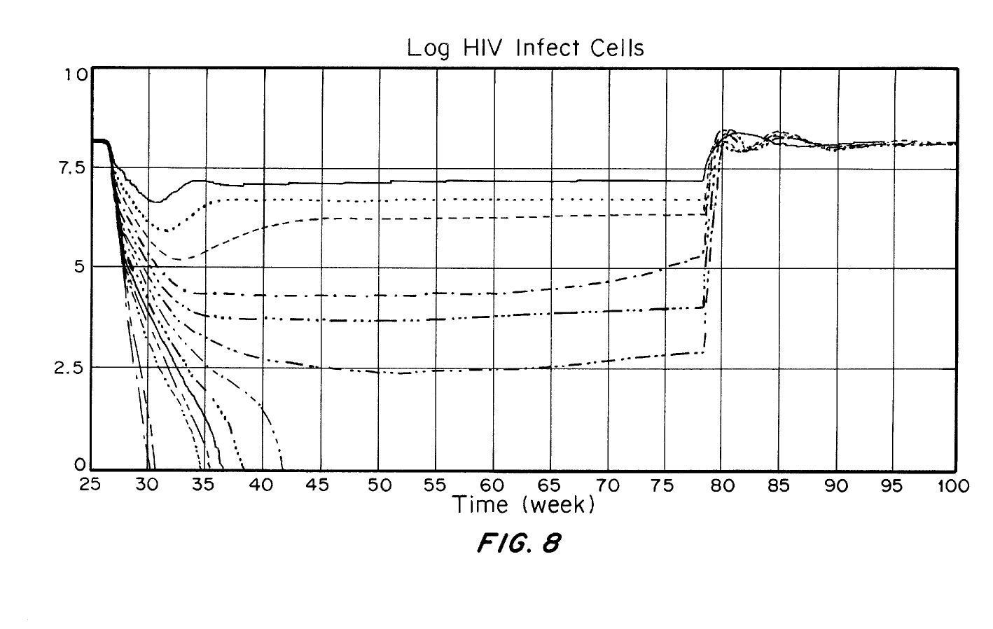

Variations on this protocol can drive simulated viral load to zero and completely eliminate the simulated virus. For example, in a protocol termed “multiple levers and HAART,” the two-lever protocol can be applied from the start of week 26 through the start of week 36 and a standard HAART protocol (two non-nucleoside reverse transcriptase inhibitors and one protease inhibitor) can be added from the start of week 34 through week 46. FIGS. 6 and 7 show the results of this protocol. In this modified protocol, viral load does not return following the termination of treatment (FIG. 6). CD4 T cell counts increase during the course of treatment and continue increasing following the termination of treatment (FIG. 7).

### Example 4

### Dependency of Results on Using Three Drugs

Treatments

The model of the human immune system can show the dependency of the results of treatment protocols on the effectiveness of the levers that are used. This example shows the dependency of HIV infected cell count on the use of three drugs, two for reduction HIV infection and one for reactivating latent HIV, in a treatment protocol. In this example treatment protocol, the two drugs for reduction of HIV infection are a CCR5 inhibitor such as Maraviroc, and anti-inflammatory, such as hydroxychloquine. Reactivating latent HIV uses a histone deacetylase inhibitor such as Vorinostat in this example treatment protocol.

All treatments were begun at week 26 and ended after week 42. Table 1 shows the drugs used in the different treatments, with an “x” indicating use of the effective amount of the drug for that row.

TABLE 1
Reduction of HIV infection with a CCR5 inhibitor; an anti-
inflammatory; and a histone deacetylase inhibitor

| Example Compound | VMC | VM | V | VC | MC | M | C |
|---|---|---|---|---|---|---|---|
| Histone deacetylase | x | X | x | x | | | |
| inhibitor (vorinostat) | | | | | | | |
| CCR5 Inhibitor | x | X | | | x | x | |
| (maraviroc) | | | | | | | |
| Chloroquine | x | | | x | x | | x |
| compound | | | | | | | |
| (hydroxychloroquine) | | | | | | | |

In the simulations, the quantity of absorbed and available drug is translated into an effect on HIV infectivity, CD4 T cell activation or reactivation of latent HIV. For Maraviroc, the infection rate was calculated as a product of the concentration of viral particles with the concentration of target cells and a rate constant. The effectiveness of Maraviroc is applied via the rate constant. For hydroxychloroquine, the priming and activation of CD4 T cells was calculated as a product of the concentration of mature antigen presenting dendritic cells, the concentration of HIV specific naïve and central memory CD4 T cells and a rate constant. The effectiveness of hydroxychloroquine is applied via the rate constant. For Vorinostat, the reactivation of latent HIV was calculated as a product of a concentration of latently infected CD4 T cells and a rate constant. The effectiveness of Vorinostat is applied via the rate constant.

Results

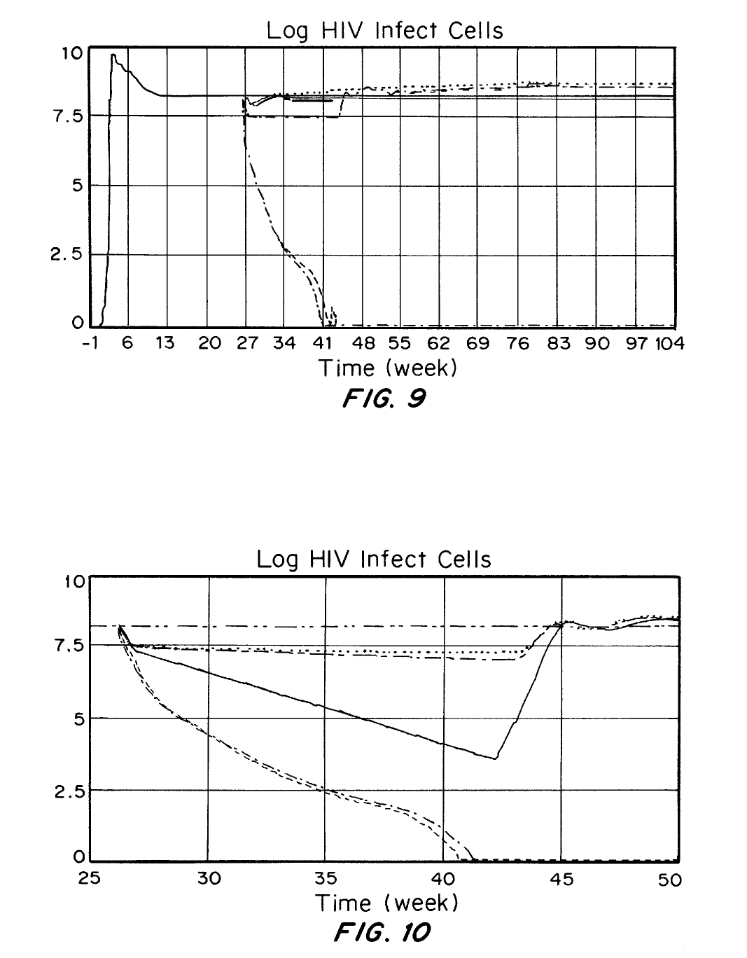

FIG. 9 displays the output from a series of simulations that include a no treatment base (only line at 20 weeks), a full treatment using effective amounts of all three drugs (VMC; lowest line at week 41), and treatments leaving one or two of the drugs out. The results show the full treatment (VMC) clears HIV infected cells by week 41. Only the treatment with both Vorinostat and Maraviroc (VM) shows clearance (second lowest line at week 41). All other treatments with only one or two of the drugs fail to clear HIV infected cells (all lines over 7.5 at week 104). In fact, all of these other treatments are essentially no better than the no treatment base. Although in these simulations hydroxychloroquine is not essential for clearance of HIV infected cells, the full treatment includes it to speed and increase the reliability of clearance.

FIG. 10 displays the output from a series of simulations that include a no treatment base (only line at 25 weeks), a full treatment using effective amounts of all three drugs (VMC; lowest line at week 41), and treatments where the effectiveness of Vorinostat is varied from the base amount. All treatments were begun at week 26 and ended after week 42. Table 2 shows the drugs used in the different treatments, with an “x” indicating use of the effective amount of the drug for that row. The number shown for Vorinostat is the rate constant used in the simulation expressed as the fold effectiveness of Vorinostat.

TABLE 2
Variable Efficacy of Vorinostat

| Example | | | | | | |
|---|---|---|---|---|---|---|
| Compound | VMC | V5MC | V4MC | V2MC | V1MC | V0.5MC |
| Histone | 3 | 5 | 4 | 2 | 1 | 0.5 |
| deacetylase | | | | | | |
| inhibitor | | | | | | |
| (vorinostat) | | | | | | |
| CCR5 Inhibitor | x | X | x | x | x | x |
| (maraviroc) | | | | | | |
| Chloroquine | x | X | x | x | x | x |
| compound | | | | | | |
| (hydroxy- | | | | | | |
| chloroquine) | | | | | | |

The results show the full treatment (VMC) clears HIV infected cells by week 41. Treatments with more effective Vorinostat (V5MC and V4MC; second lowest lines at week 41 (the lines are overlapping)) also clear HIV infected cells, but slightly slower than the VMC treatment. This is one reason why the amount of Vorinostat used for the full treatment (VMC) was chosen. The other treatments with less than the effective amount of Vorinostat fail to clear HIV infected cells during the treatment period (V2MC, V1MC, and V0.5MC; all lines over 7.5 at week 50). All of these other treatments are essentially no better than the no treatment base by week 50.

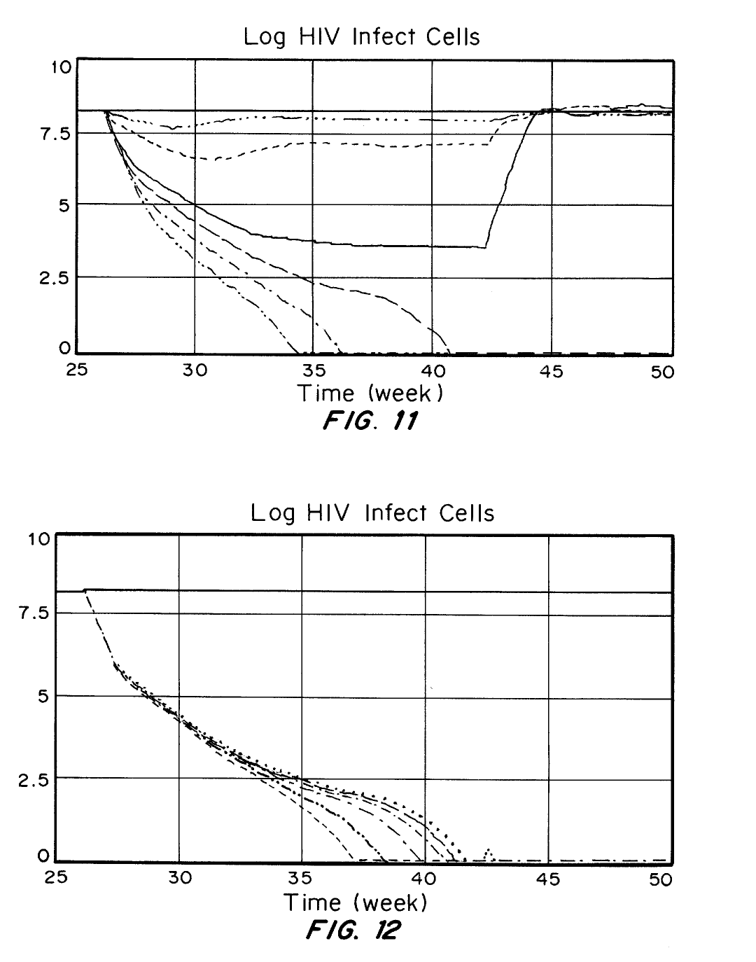

FIG. 11 displays the output from a series of simulations that include a no treatment base (only line at 25 weeks), a full treatment using effective amounts of all three drugs (VMC; line that goes to zero at week 41), and treatments where the effectiveness of Maraviroc is varied from the base amount. All treatments were begun at week 26 and ended after week 42. Table 3 shows the drugs used in the different treatments, with an “x” indicating use of the effective amount of the drug for that row. The number shown for Maraviroc is the rate constant used in the simulation expressed as the fold effectiveness of Maraviroc.

TABLE 3
Variable Efficacy of Maraviroc

| Example Compound | VMC | VM3C | VM2.5C | VM1.5C | VM0.5C | VM0.1C |
|---|---|---|---|---|---|---|
| Histone deacetylase | x | X | x | x | x | x |
| inhibitor (vorinostat) | | | | | | |
| CCR5 Inhibitor | −2 | −3 | −2.5 | −1.5 | −0.5 | −0.1 |
| (maraviroc) | | | | | | |
| Chloroquine | x | X | x | x | x | x |
| compound | | | | | | |
| (hydroxychloroquine) | | | | | | |

The results show the full treatment (VMC) clears HIV infected cells by week 41. Treatments with more effective Maraviroc (VM3C and VM2.5C; lines that go to zero at weeks 34 and 36, respectively) also clear HIV infected cells. A lower effectiveness of Maraviroc was assumed for the full treatment (VMC) because of the uncertainty and variability of actual Maraviroc effectiveness. The other treatments with a less effective Maraviroc fail to clear HIV infected cells (VM1.5C, VM0.5C, and VM0.1C; all lines over 7.5 at week 50). All of these other treatments are essentially no better than the no treatment base by week 50.

FIG. 12 displays the output from a series of simulations that include a no treatment base (only line at 25 weeks), a full treatment using effective amounts of all three drugs (VMC; line that goes to zero at week 41), and treatments where the effectiveness of hydroxychloroquine is varied from the base amount. All treatments were begun at week 26 and ended after week 42. Table 4 shows the drugs used in the different treatments, with an “x” indicating use of the effective amount of the drug for that row. The number shown for hydroxychloroquine is the rate constant used in the simulation expressed as the fold effectiveness of hydroxychloroquine.

TABLE 4
Variable efficacy of Hydroxychloroquine

| Example Compound | VMC | VMC0.6 | VMC0.4 | VMC0.2 | VMC0.05 | VMC0.01 |
|---|---|---|---|---|---|---|
| Histone deacetylase | x | X | x | x | x | x |
| inhibitor (vorinostat) | | | | | | |
| CCR5 Inhibitor | x | X | x | x | x | x |
| (maraviroc) | | | | | | |
| Chloroquine | −0.1 | −0.6 | −0.4 | −0.2 | −0.05 | −0.01 |
| compound | | | | | | |
| (hydroxychloroquine) | | | | | | |

The results show the full treatment (VMC) clears HIV infected cells by week 41. Treatments with more hydroxychloroquine (VMC0.6, VMC0.4 and VMC0.2; lines that go to zero at weeks 37, 38, and 39, respectively) also clear HIV infected cells. A lower effectiveness of hydroxychloroquine was chosen for the base treatment (VMC) because of the significant uncertainty around the actual effectiveness of hydroxychloroquine in reducing CD4+T cell activation. The treatments with less than the effective amount of hydroxychloroquine clears HIV infected cells by week 42 (VMC0.05; line that goes to zero at week 42; VMC0.01; line that goes to zero after week 43).

### Example 6

### Clinical Protocol for Treatment of HIV

Study Title:

A randomized study to compare the efficacy of vorinostat/hydroxychloroquine/maraviroc (VHM) in controlling HIV after treatment interruption in subjects who initiated ART during acute HIV infection (SEARCH 019)

Institution Name:

The Thai Red Cross AIDS Research Centre, Bangkok, Thailand

Primary Objective

To compare the proportion of patients between vorinostat/hydroxychloroquine/maraviroc (VHM) co-administered with anti-retroviral therapy (ART) versus ART only arms who are able to maintain HIV RNA <50 copies/ml following treatment interruption.

Secondary Objectives

Time to HIV RNA rebound after treatment interruption between VHM+ART versus ART only arms;

To compare the cell-associated HIV RNA (multispliced and unspliced) in total CD4 T cells between the VHM+ART versus ART only arms;

To compare markers of HIV persistence (total and integrated HIV DNA and 2-LTR circles) between the VHM+ART versus ART arms;

To compare histone acetylation (H3) between the VHM+ART versus ART arms;

To compare adverse events both related and unrelated to the combination of vorinostat, hydroxychloroquine and maraviroc between arms;

To compare the occurrence and severity of acute retroviral syndrome between arms following treatment interruption;

To prospectively validate the simulation model of a functional cure for HIV-1 infection.

Hypotheses:

A higher proportion of patients with HIV RNA <50 copies/ml following treatment interruption at the end of the study;

Longer time to HIV RNA rebound following treatment interruption;

Higher cell-associated RNA in total CD4 T cells at the end of the VHM treatment period;

Lower reservoir size and 2 LTR circles at the end of VHM treatment period and the end of the study;

Higher H3 acetylation at the end of VHM treatment;

Higher adverse events related to VHM;

Similar rates of acute retroviral syndrome after treatment interruption in subjects experiencing viral rebound.

This will be a single-center proof-of-concept study in which recruitment and follow-up of volunteers will be done at the Thai Red Cross AIDS Research Centre (TRC-ARC). The TRC-ARC has extensive experience in executing clinical HIV treatment studies with intensive specimen collections, processing, storage, international shipments and complex laboratory assays. The TRC-ARC is associated with two internationally-accredited (College of American Pathologists) clinical laboratory facilities.

Study Design

An exploratory, open label, randomized study of Vorinostat/Hydroxychloroquine/HAART versus HAART only.

Study Participants

Subjects will be recruited from RV254/SEARCH 010. RV254/SEARCH 010 is an acute HIV infection cohort funded by the US Military HIV Research Program and conducted by the TRC-ARC in Bangkok, Thailand. Subjects will be co-enrolled in RV254/SEARCH 010 but will not have any blood drawn for RV254/SEARCH 010 during the period of co-enrollment, so the total blood draw in this treatment interruption study represents the only blood samples that will be taken from these patients.

Extensive feasibility data exists for enrolling and retaining subjects. Screening for acute HIV infection in RV254/SEARCH 010 is performed in real-time by pooled nucleic acid testing and sequential enzyme immunoassay and Western blot assay. Since April 2009, the study has screened more than 55,000 samples and identified over 100 subjects with acute HIV infection. These subjects have been classified using the Fiebig and 4thG staging systems for acute HIV infection, and for this study we propose to use acutely infected subjects who were staged as Fiebig III or later. Subjects aged 18-60 years old, who initiated ART during acute HIV infection stages and have maintained viral suppression (HIV RNA <50 copies/ml) for at least the prior 28 weeks will be asked to enroll in the study. The subjects must have CD4>450 cells/μl, and EKG and laboratory values within acceptable ranges. Subjects positive for HBsAg or with malignancy will be excluded. It is anticipated that over 85% of subjects in this study will be male as reflected by the RV254/SEARCH010 study population.

Sample Size

Fifteen subjects will be enrolled randomized 2:1 to VHM (N=10) vs HAART (N=5) only.

Study Drug

Vorinostat will be administered at 400 mg orally every 24 h for 3 cycles, each of 14 days with an interim rest-period of 14 days between cycles. HCQ will be administered at a dose of 200 mg 2×/daily during the course of vorinostat administration (10 weeks). Maraviroc will be administered at 600 mg 2×/daily on the same schedule as HCQ. This dose of maraviroc is based on its concomitant use with efavirenz. Dosing will be adjusted as appropriate should the subject be on an integrase inhibitor or a protease inhibitor instead of efavirenz due to intolerance to the drug or primary NNRTI resistance. Any standard ART may be used. However, it is expected that the majority of subjects will be on 2 nucleos(t)ide reverse-transcriptase inhibitors (NRTI) [emtricitabine (FTC) and tenofovir (TDF) and 1 non-nucleoside reverse transcriptase inhibitor [efavirenz (EFV)] to all study participants at the following dosage: FTC, 200 mg 1×/day or 3TC, 300 mg 1×/day; TDF, 300 mg 1×/day and EFV, 600 mg 1×/day.

In subjects on NNRTI-based therapy, the NNRTI will be interrupted at week 8 and the rest of the regimens will be interrupted at week 10. In order to prevent NNRTI resistance, protease inhibitor replacement therapy with darunavir 900 mg 1×/day with ritonavir 100 mg 1×/day will be given between weeks 8 and 10 and maraviroc will be reduced from 1200 mg/day to 600 mg/day., 200 mg 1×/day; TDF, 300 mg 1×/day and EFV, 600 mg 1×/day.

Study Duration

A minimum of 34 weeks and up to 80 weeks: Subjects must have been on ART for a minimum of 42 weeks prior to study entry. Note that some subjects may be enrolled from RV254/SEARCH010 who have already fulfilled the minimum 42-week ART requirement. The VHM treatment will occur over 10 weeks and the follow-up period will be 24 weeks.

1. A method of preventing or delaying a rise in viral load following cessation of treatment of human immunodeficiency virus (HIV) infection, the method comprising:
administering to a subject infected with HIV at least three compounds collectively having the following activities:
dampening of immune activation, wherein the dampening selectively affects the CD4 T cell response relative to the CD8 T cell response,
inhibition of HIV replication,
stimulation of reactivation of latent HIV, and
inhibition of infection of CD4 T cells by HIV, wherein the compound that inhibits HIV infection of CD4 T cells is selected from the group consisting of C-C chemokine receptor type 5 (CCR5) inhibitors, C-X-X chemokine receptor type 4 (CXCR4) inhibitors, CD4 inhibitors, gp120 inhibitors, and gp41 inhibitors,
wherein the compounds are provided in dosages reducing the number of cells infected with HIV or the viral load of HIV, relative to which is achieved using just the combination of ART and compounds which activate latent HIV,
wherein HIV infected cells or HIV viral load is not detectable at 12 months after the end of the course of treatment.

2. The method of claim 1 wherein the compound that inhibits HIV replication are selected from the group consisting of nucleoside reverse transcriptase inhibitors (NRTIs) such as tenofovir, emtricitabine, zidovudine (AZT), lamivudine (3TC), abacavir, and tenofovir alafenamide fumarate; non-nucleotide reverse transcriptase inhibitors (NNRTIs) such as efavirenz, rilpivirine, and etravirine; integrase inhibitors such as raltegravir and elvitegravir; and protease inhibitors such as ritonavir, darunavir, atazanavir, lopinavir, and cobicistat.

3. The method of claim 1 wherein the compound that dampens immune activation is selected from the group consisting of anti-inflammatories such as hydroxychloroquine, chloroquine, PD-1 inhibitors, type I interferons, IL6, cyclo-oxygenase-2 inhibitors, peroxisome proliferator-activated receptor-c (PPAR-c) agonists such as pioglitazone and leflunomide, methotrexate, mesalazine, and anti-fibrotic agents such as angiotensin-converting enzyme (ACE) inhibitors.

4. (canceled)

5. The method of claim 1 further comprising administering a stimulator of CD8 T cell response to HIV such as IL-2, IL-12, IL-15, or a combination thereof, or a composition that stimulates production in the subject of IL-2, IL-12, IL-15, or a combination thereof.

6. The method of claim 1 wherein the compound that stimulates reactivation of latent HIV is selected from the group consisting of histone deacetylase (HDAC) inhibitors such as vorinostat, romidepsin, pomidepsin, panpbinostat, givinostat, belinostat, valproic acid, CI-994, MS-275, BML-210, M344, NVP-LAQ824, mocetinostat, and sirtuin inhibitors; NF-κB-inducing agents such as anti-CD3/CD28 antibodies, tumor necrosis factor alpha (TNFα), prostratin, ionomycin, bryostatin-1, and picolog; histone methyltransferase (HMT) inhibitors such as BIX-01294 and chaetocin; pro-apoptotic and cell differentiating molecules such as JQ1, nutlin3, disulfiram, aphidicolin, hexamethylene bisacetamide (HMBA), dactinomycin, aclarubicin, cytarabine, Wnt small molecule inhibitors, Notch inhibitors; immune modulators such as anti-PD-1 antibodies, anti-CTLA-4 antibodies, anti-TRIM-3 antibodies, and BMS-936558; and CD4 T cell vaccines.

7. The method of claim 1 wherein the compound that inhibits HIV infection of CD4 T cells is a CCR5 inhibitor selected from the group consisting of maraviroc, aplaviroc, vicriviroc, TNX-355, PRO 140, BMS-488043, plerixafor, epigallocatechin gallate, anti-gp120 antibody, such as antibody b12, griffithsin, DCM205, and Designed Ankyrin Repeat Proteins (DARPins).

8. The method of claim 7, wherein a dosage of CCR5 inhibitor equivalent to 200 to 600 mg of Maraviroc is administered per day.

9. The method of claim 3, wherein the compound that dampens immune activation is a chloroquine or hydroxychloroquine.

10. The method of claim 9, wherein the chloroquine or hydroxychloroquine is administered in a dosage equivalent to hydroxychloroquine in a dosage of between 150 to 400 mg administered per day.

11. The method of claim 6 wherein the compound that stimulates reactivation of latent HIV comprises a histone deacetylase inhibitor.

12. The method of claim 11, wherein the histone deacetylase inhibitor is administered in a dosage equivalent to Vorinostat at a dosage of from 150 to 400 mg administered per day.

13. The method of claim 1, wherein:
the compound that inhibits HIV infection of CD4 T cells is a CCR5 inhibitor such as Maraviroc,
the compound that dampens immune activation is an anti-inflammatory compound such as hydroxychloroquine, and
the compound that stimulates reactivation of latent HIV is a histone deacetylase inhibitor such as Vorinostat.

14. The method of claim 13 further comprising administering HART.

15. The method of claim 14 comprising administering:
Vorinostat at a dosage of 400 mg orally every 24 hours for 2 cycles of 14 days with an interim rest-period of 14 days between cycles;
Hydroxychloroquine (H) at a dosage of 200 mg twice daily during the course of vorinostat administration with no rest-period during the interim cycle;
Maraviroc (M) at a dosage of 600 mg twice daily during the course of vorinostat administration with no rest-period during the interim cycle; and
HAART in the form of two nucleos(t)ide reverse-transcriptase inhibitors such as emtricitabine (FTC) and tenofovir (TDF) and one non-nucleoside reverse transcriptase inhibitor such as efavirenz (EFV) for the duration of the treatment at a dosage equivalent to FTC, 200 mg 1×/day; TDF, 300 mg 1×/day and EFV, 600 mg 1×/day.

16. The method of claim 1, wherein the compounds are administered for a period of time from 10 weeks to 40 weeks or at least two weeks after HIV infected cells or HIV viral load becomes undetectable.

17. The method of claim 1, wherein the subject has not been administered any anti-HIV treatment for at least 10 weeks prior to administration of the inhibitors and reactivation stimulator.

18. (canceled)

19. The method of claim 1, wherein HIV infected cells or HIV viral load is not detectable at 12 months after the end of the course of treatment.

20. A composition for use in the method of claim 1.

21. The method of claim 1, wherein the subject has not been administered any anti-HIV treatment for at least two weeks prior to administration of the inhibitors and reactivation stimulator.

22. A combination of separate compositions for *preventing or delaying a rise in viral load following cessation of treatment of human immunodeficiency virus (HIV) infection, the combination comprising at least three compounds collectively having the following activities:
dampening of immune activation, wherein the dampening selectively affects the CD4 T cell response relative to the CD8 T cell response,
inhibition of HIV replication,
stimulation of reactivation of latent HIV, and
inhibition of infection of CD4 T cells by HIV, wherein the compound that inhibits HIV infection of CD4 T cells is selected from the group consisting of C-C chemokine receptor type 5 (CCR5) inhibitors, C-X-X chemokine receptor type 4 (CXCR4) inhibitors, CD4 inhibitors, gp120 inhibitors, and gp41 inhibitors,
wherein the compounds are provided in dosages reducing the number of cells infected with HIV or the viral load of HIV, relative to which is achieved using just the combination of ART and compounds which activate latent HIV,
wherein each separate composition comprises one or more of the at least three compounds.

23. The combination of claim 22, wherein the combination comprises:
Vorinostat (V) at a dosage of 400 mg in one of the separate compositions;
Hydroxychloroquine (H) at a dosage of 200 mg in one of the separate compositions;
Maraviroc (M) at a dosage of 600 mg in one of the separate compositions;
a nucleos(t)ide reverse-transcriptase inhibitor at a dosage equivalent to 200 mg of emtricitabine (FTC) in one of the separate compositions;
a nucleos(t)ide reverse-transcriptase inhibitor at a dosage equivalent to 300 mg of tenofovir (TDF) in one of the separate compositions; and
a non-nucleoside reverse transcriptase inhibitor at a dosage equivalent to 600 mg of efavirenz (EFV) in one of the separate compositions.
